(19) United States
(12) Patent Application Publication
(10) Pub. No.: US 2026/0183432 A1
(43) Pub. Date: Jul. 2, 2026

(54) IMAGING AGENTS FOR DETECTING CD206+ MACROPHAGES

(71) Applicant: THE GENERAL HOSPITAL CORPORATION, Boston, MA (US)

(72) Inventors: Cuihua Wang, Cambridge, MA (US); John W. Chen, Newton, MA (US)

(21) Appl. No.: 19/129,569

(22) PCT Filed: Nov. 15, 2023
(86) PCT No.: PCT/US2023/079802

§ 371 Date: May 13, 2025

Related U.S. Application Data
(60) Provisional application No. 63/383,967, filed on Nov. 16, 2022.

(51) Int. Cl. A61K 49/10; A61K 31/7008; A61K 31/7056; A61K 31/7135; A61K 49/00; C07H 15/26; C07H 23/00; C12N 5/0786; G01N 33/50

(52) U.S. Cl. CPC A61K 49/106; A61K 31/7008; A61K 31/7056; A61K 31/7135; A61K 49/0002; A61K 49/0021; C07H 15/26; C07H 23/00; C12N 5/0645; G01N 33/5005

(57) ABSTRACT

This disclosure relates to imaging agents such as compounds of Formula (I): or pharmaceutically acceptable salts thereof, for the detection of CD206⁺ macrophages in vitro and in vivo, wherein the variables are described herein.

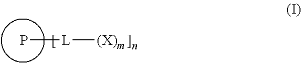

### RELATED APPLICATION DATA

The present disclosure claims the priority of U.S. application No. 63/383,967, filed Nov. 16, 2022, which is hereby incorporated by reference in its entirety.

### STATEMENT OF GOVERNMENT SUPPORT

This invention was made with government support under K25 HL150305, R01 NS103998, RF1 AG075055 awarded by the National Institute of Health, RG-1902-33633 from the National Multiple Sclerosis Society, and the National Academy of Medicine. The government has certain rights in the invention.

### FIELD

This disclosure relates to imaging agents for the detection of CD206⁺ macrophages in vitro and in vivo.

### BACKGROUND

Macrophages are key drivers of the innate immune response and different subtypes exist in vitro and in vivo. Two major subsets of activated macrophages are M1 and M2. Different populations of activated macrophages play distinct roles and can adapt different phenotypes in response to new microenvironmental stimuli in various pathophysiological processes such as wound healing, cancer, and myocardial infarction, among others. Reprogramming to shift between phenotypes has become a promising strategy, especially in tumor-associated macrophages (TAMs)-targeted immunotherapies. These emerging therapies targeting activated macrophages promote the need for noninvasive methods mapping and tracking of CD206⁺ macrophages to predict therapeutic outcomes.

### SUMMARY

Activated macrophages play key while distinctive roles in response to different pathophysiological conditions. However, specific and noninvasive MRI agents to detect CD206⁺ macrophages have not been available. We developed fluorescent (MR2-cy5) and MRI agents (Mann2-DTPA-Gd and MannGdFish) that target the mannose receptor (CD206), a surface marker expressed by anti-inflammatory/reparative macrophages and demonstrated that these agents are specific to CD206⁺ macrophages both in vitro and in vivo. MRI of Mann2-DTPA-Gd and MannGdFish can track the evolution of reparative inflammation in cutaneous wound healing and detect tumor-associated macrophages (TAMs) in glioma. Importantly, MannGdFish, with its high sensitivity, specificity, stability, and favorable biodistribution and pharmacokinetics, is a promising translational candidate to noninvasively monitor CD206⁺ macrophages in repair/regeneration and tumor in patients.

Some embodiments provide a compound of Formula (I)

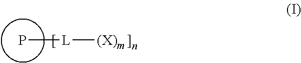

or a pharmaceutically acceptable salt thereof, wherein
P is a fluorescence imaging probe or a magnetic resonance imaging probe;
m is 1, 2, 3, 4, 5, 6, 7, 8, 9, 10, 11, or 12;
each X is independently X-1 or X-2:

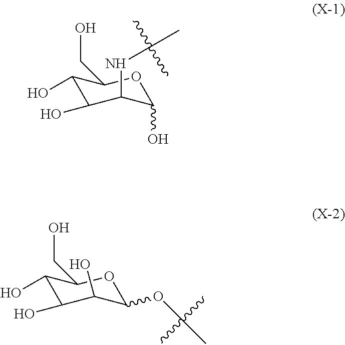 [SMILES: *NC1C(O)OC(CO)C(O)C1O ; *OC1OC(CO)C(O)C(O)C1O]

L is a C2-C20 alkylene; and
n is 1, 2, 3, 4, 5, or 6.

Some embodiments provide a pharmaceutical composition comprising a compound of Formula (I), or a pharmaceutically acceptable salt thereof, and a pharmaceutically acceptable carrier.

Some embodiments provide a method of detecting M2-like macrophages in vitro, the method comprising:
(i) contacting a compound of Formula (I) with cells or tissues;
(ii) waiting a time to allow the compound to accumulate in the cells or tissues; and
(iii) acquiring an image of the cells or tissues.

Some embodiments provide a method of monitoring M2-like macrophages in an organ or a tissue of a subject, the method comprising:
(i) administering to the subject an effective amount of a compound of Formula (I), or a pharmaceutically acceptable salt thereof, or a pharmaceutical composition as described herein;
(ii) waiting a time sufficient to allow the compound to accumulate in the organ or a tissue to be imaged; and
(iii) acquiring an image of the organ or the tissue of the subject,
wherein observing the image attributable to the compound of Formula (I) is indicative of M2-like macrophages in an organ or tissue.

Some embodiments provide a method of monitoring treatment of a disease or condition in a subject, the method comprising:
(i) administering to the subject an effective amount of a compound of Formula (I), or a pharmaceutically acceptable salt thereof, or a pharmaceutical composition as described herein;
(ii) waiting a time sufficient to allow the compound to accumulate in an organ or tissue of the subject affected by the disease or condition;
(iii) acquiring a first image of the organ or the tissue of the subject;
(iv) administering to the subject a therapeutic agent in an effective amount to treat the disease or condition;
(v) administering to the subject an effective amount of a compound of Formula (I), or a pharmaceutically acceptable salt thereof, or a pharmaceutical composition as described herein;
(vi) waiting a time sufficient to allow the compound to accumulate in the organ or tissue of the subject affected by the disease or condition;
(vii) acquiring a second image of the organ or the tissue of the subject; and
(viii) comparing the first and the second images,
wherein observing a difference between the first and the second images attributable to the compound of Formula (I) is indicative of progression of treatment of the disease or condition.

Some embodiments provide a method of diagnosing a disease or condition in a subject, the method comprising:
(i) administering to the subject an effective amount of a compound of Formula (I), or a pharmaceutically acceptable salt thereof, or a pharmaceutical composition as described herein;
(ii) waiting a time sufficient to allow the compound to accumulate in an organ or a tissue of the subject affected by the disease or condition; and
(iii) acquiring an image of the organ or the tissue of the subject,
   wherein observing the image attributable to the compound of Formula (I) is indicative of the disease or condition.

Some embodiments provide a method of intraoperative imaging in a subject, the method comprising:
(i) administering to the subject an effective amount of a compound of Formula (I), or a pharmaceutically acceptable salt thereof, or a pharmaceutical composition as described herein;
(ii) waiting a time sufficient to allow the compound to accumulate in an organ or a tissue of the subject affected by the disease or condition; and
(iii) acquiring an image of the organ or the tissue of the subject,
   wherein observing the image attributable to the compound of Formula (I) defines margins.

The details of one or more embodiments of the subject matter of this disclosure are set forth in the accompanying drawings and the description. Other features, aspects, and advantages of the subject matter will become apparent from the description, the drawings, and the claims.

### BRIEF DESCRIPTION OF DRAWINGS

FIG. 1 shows the chemical structures of MR1-cy5, MR2-cy5, Mann2-DTPA-Gd, and MannGdFish.

FIG. 2A shows fluorescence imaging of MR1-cy5 incubated with M1 and M2 macrophages at 37° C. showed low specificity to M2 macrophages, and FIG. 2B shows MR2-cy5 fluorescence imaging of M1 and M2 macrophages demonstrating much higher signal from M2 macrophages than that from M1 macrophages and higher specificity to M2 macrophages.

FIG. 3 shows the amount of gadolinium (detected by inductively coupled plasma mass spectrometry (ICP-MS)) on M2 macrophages was much higher than that on M1 macrophages and HEK293 cells (as non-specific control) when incubated with Mann2-DTPA-Gd (M2 vs. M1 with probe, one-way ANOVA. n=4 for M1 and M2 macrophages and n=3 for HEK293 cells. ****, p<0.0001).

FIG. 4 shows MR images of Mann2-DTPA-Gd at 15 min and 60 min in wildtype and mannose-receptor knock-out mice, respectively in mouse model of subcutaneous wound healing.

FIG. 5 shows the MR contrast-to-noise ratio (CNR) of mannose receptor knock-out mice decreased significantly after 15 min post-injection compared to that in wildtype mice on day 7 (n=3, Mann2-DTPA-Gd administration at 0.3 mmol/kg mouse) in mouse model of subcutaneous wound healing.

FIG. 6 shows MR imaging of Mann2-DTPA-Gd in a mouse model of subcutaneous wound healing. Longitudinal MR imaging of Mann2-DTPA-Gd at 60 min post-injection showed that CNR on day 7 was much higher compared to that on days 1 and 4.

FIG. 7 shows the percentage of YFP+ cells (arginase-1-positive cells, a marker for M2-like cells) on day 7 was significantly higher than that on day 1 and day 4 (n=3, One-way ANOVA. ****, p<0.0001) in a mouse model of subcutaneous wound healing.

FIG. 8 shows MR imaging of glioma with Mann2-DTPA-Gd. MR imaging of glioma on the third week for pre-contrast, and at 30 and 60 min post-injection of Mann2-DTPA-Gd.

FIG. 9 shows cytotoxicity of MannGdFish in an MTT assay. No cytotoxicity was observed at a concentration as high as 5 mM (n=3).

FIG. 10 shows biodistribution of MannGdFish at 3 h and on day 7 after MannGdFish injection (24 hours after wound induction) detected by ICP-MS in a mouse model of cutaneous wound healing.

FIG. 11 shows inductively coupled plasma mass spectrometry (ICP-MS) of wound skin compared to normal skin demonstrated the accumulation of MannGdFish in wound skin at 3 h and day 7 (two-way ANOVA, ns: no significant difference, p=0.10; **, p=0.037).

FIG. 12 shows representative MR images of MannGdFish and DOTA-Gd in precontrast and at 60 min. Signal of MannGdFish was much higher in comparison with that of DOTA-Gd in wound healing at 60 min.

FIG. 13 shows MR imaging of MannGdFish demonstrated slightly higher CNRs and similar dynamic profile as that of Mann2-DTPA-Gd in wildtype wound healing mice on day 7, Both of which showed slow decrease after 45 min while CNR of DOTA-Gd imaging decreased significantly 15 min post-injection.

FIG. 14 shows the high resolution mass spectrum of MR1-cy5.

FIG. 15 shows the high resolution mass spectrum of MR2-cy5.

FIG. 16 shows the high resolution mass spectrum of Mann2-DTPA-Gd.

FIG. 17A shows relaxivity (r1) of Mann2-DTPA-Gd, and FIG. 17B shows validation of differentiation of M1 and M2 macrophages.

FIG. 18A shows contrast-to-noise ratio (CNR) of wildtype mouse at 45 min post-injection on day 7 was about two-fold higher compared to that on day 4 after wound injury, and FIG. 18B shows the percentage of CD86+ cells (a marker for M1-like cells) on day 4 was significantly higher than that on day 1 and day 7 (n=3, One-way Anova, **, p=0.005 and p=0.001, respectively, for day 1 and day 7).

FIG. 19 shows the high resolution mass spectrum of MannGdFish.

FIG. 20A shows Relaxivity (r1) of MannGdFish, and FIG. 20B shows the blood half-life of MannGdFish detected by ICP-MS using a two-way exponential model was 0.3 min for the fast phase and 6.1 min for the slow phase.

FIG. 21A shows conditions for preparation of PET probe via chelation of [68]Ga with MannGdFish. FIG. 21B shows radio-HPLC of [68]Ga-MannGaFish indicating clean labeling with high purity (>95%). FIG. 21C shows ex vivo biodistribution of [68]Ga-MannGaFish with gamma-counting. FIG. 21D shows representative images of [68]Ga-MannGaFish PET imaging indicating low uptakes in the lungs and heart.

FIG. 22 shows additional multi-mannose imaging agents of Formula (I).

### DETAILED DESCRIPTION

Activated macrophages play important roles in the innate immune response, and their diversity drives both damage and repair in many diseases. The development of non-invasive imaging technologies to differentiate between the different subtypes of activated macrophages is critical to better understand the functions of these cells in diseases and to develop novel therapies targeting subtypes of macrophages. Here, we developed fluorescent and MRI agents to detect anti-inflammatory/reparative macrophages by targeting the mannose receptor (CD206) and validated the specificity and efficacy of the agents both in vitro in cellular assays and in vivo in animal models of cutaneous wound healing and glioma.

Featured herein are fluorescent (MR2-cy5) and MRI agents (Mann2-DTPA-Gd and MannGdFish) that target the mannose receptor (CD206), a surface marker expressed by anti-inflammatory/reparative macrophages. In some embodiments, these agents are specific to CD206⁺ macrophages both in vitro and in vivo. In some embodiments, the MRI of Mann2-DTPA-Gd and MannGdFish can track the evolution of reparative inflammation in cutaneous wound healing and detect tumor-associated macrophages (TAMs) in glioma. In some embodiments, the MRI agent is MannGdFish, which demonstrates high sensitivity, specificity, stability, and favorable biodistribution and pharmacokinetics; as such, MannGdFish represents a promising translational candidate to noninvasively monitor CD206⁺ macrophages in repair/regeneration and tumor in patients.

### Definitions

As used herein, “DOTA-Gd” refers to a compound having the structure:

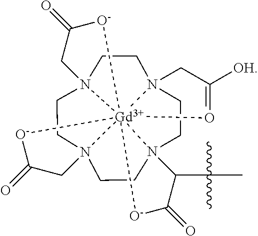

As used herein, the term “alkylene” refers to a straight or branched divalent hydrocarbon chain that comprises a fully saturated (no double or triple bonds) hydrocarbon group. Alkylene groups may have 1 to 20 carbon atoms (whenever it appears herein, a numerical range such as “1 to 20” refers to each integer in the given range; e.g., “1 to 12 carbon atoms” means that the alkylene group may consist of 1 carbon atom, 2 carbon atoms, 3 carbon atoms, etc., up to and including 12 carbon atoms). The alkylene group of the compounds may be designated as “C1-C3 alkylene” or similar designations. By way of example only, “C1-C3 alkylene” indicates that there are one to four carbon atoms in the alkylene chain, i.e., the alkylene chain is selected from methylene, ethylene, propylene, and iso-propylene. Alkylene groups optionally include 1-6 oxo (C═O) groups, and are optionally interrupted by 1-6 heteroatoms independently selected from N and O, valence and chemical stability permitting, and are optionally interrupted by up to one phenyl group. In some embodiments, the alkylene groups described herein are interrupted by one or two amide groups (—NH(C═O)— or —(C═O)NH—). In some embodiments, when the alkylene group is interrupted by a phenyl group, the phenyl serves as a branch point in the alkylene, as shown herein.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “imaging effective amount” refers to the amount of active compound that can image a tissue, system, animal, individual or human that is being sought by a researcher, veterinarian, medical doctor or other clinician under standard imaging conditions.

The terms “chelating ligand,”, “chelating backbone”, and “chelate moiety” are used interchangeably and refer to any polydentate ligand that is capable of coordinating a metal ion, either directly or after removal of protecting groups, or is a reagent, with or without suitable protecting groups, that is used in the synthesis of a MR contrast agent and comprises substantially all of the atoms that ultimately will coordinate the metal ion of the final metal complex. The terms “chelate” or “metal chelate” refer to the actual metal-ligand complex. It is understood that the polydentate ligand can eventually be coordinated to a medically useful or diagnostic metal ion.

Coordination of metal ions by water and other ligands is often regarded in terms of coordination spheres (see e.g., D. T. Richens, The Chemistry of Aqua Ions, John Wiley and Sons, New York, 1997, Chapter 1). The first or primary coordination sphere represents all the ligands directly bonded to the metal ion and is defined by the ligands. There is a second coordination sphere where water molecules and counterions bond to the groups in the first coordination sphere via hydrogen bonding and electrostatic interactions. Tertiary and subsequent coordination spheres are typically termed “bulk water” or “bulk solvent”. The distinctions between these spheres are both spatial and temporal. The first coordination sphere is typically well-defined and the time that a water or other ligand spends in the first coordination sphere is longer than in other coordination spheres. The second sphere is less well-defined, but the waters here have a longer lifetime than the typical diffusion time of water. Beyond the second sphere water diffuses freely.

The term “fluorescence imaging probe” as used herein refers to a moiety capable of fluorescence emission from 400 nm to 1000 nm, which is ideally bright, displays a high a signal-to-noise ratio, and with limited photobleaching.

The term “magnetic resonance imaging probe” as used herein refers to a moiety capable of creating a hyperintense contrast (brightening the tissue of interest) via T1 (spin-lattice) relaxation time or T2 spin-spin relaxation, changing the relaxation rate of protons in water, creating a change in signal on MRI.

The term “specific binding affinity” as used herein, refers to the capacity of a contrast agent to be taken up by, retained by, or bound to a particular or target biological component to a greater degree as compared to other non-targeted biological components. Contrast agents that have this property are said to be “targeted” to the “target” component. Contrast agents that lack this property are said to be “non-specific” or “non-targeted” agents. The binding affinity of a binding group for a target is expressed in terms of the equilibrium dissociation constant “K_{d}.”

The term “relaxivity” as used herein, refers to the increase in either of the MR quantities 1/T₁ or 1/T₂ per millimolar (mM) concentration of paramagnetic ion or contrast agent, which quantities may be different if the contrast agent contains a multiplicity of paramagnetic ions, wherein T₁ is the longitudinal or spin-lattice relaxation time, and T₂ is the transverse or spin-spin relaxation time of water protons or other imaging or spectroscopic nuclei, including protons found in molecules other than water. Relaxivity is expressed in units of mM⁻¹ s⁻¹.

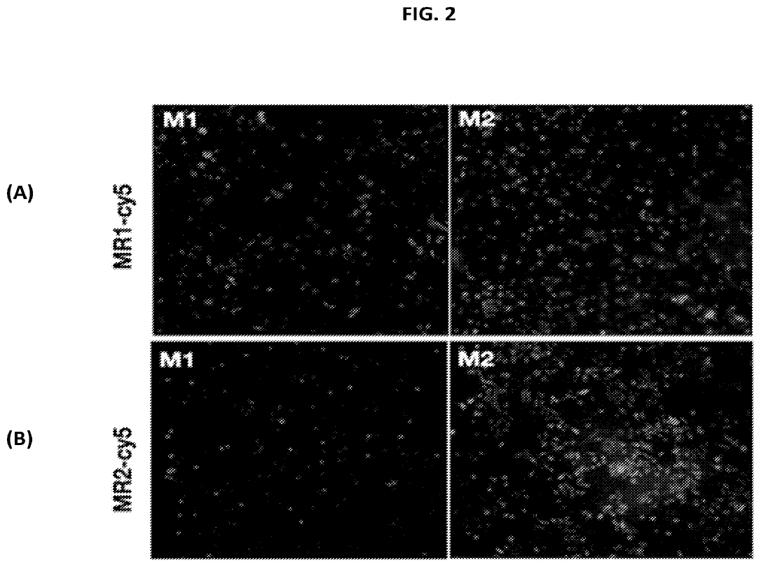

Mannose receptor (CD206) is a well-established anti-inflammatory/reparative macrophage surface biomarker: CD206 is a 175 kDa transmembrane protein that recognizes and mediates endocytosis of pathogens by binding to glycoproteins terminated with mannose, fucose or N-acetyl-glucosamine. Elevated expression of CD206 has been found in anti-inflammatory/reparative macrophages in various pathophysiological conditions and has become an emerging therapeutic target. Imaging agents targeting CD206 to detect anti-inflammatory/reparative macrophages have also been reported using either D-mannose-based agents or CD206-specific antibody/nanobody based SPECT/PET imaging. However, in addition to radiation exposure concerns, these agents have issues with specificity. D-mannose has a relatively low binding affinity to CD206 compared to its oligomers and clustered analogs (IC₅₀: 5.5 mM vs. 18-23 μM or more. Furthermore, no work has been conducted to evaluate the potency of these clustered mannosides as MR imaging agents in part due to the challenges in synthesizing these carbohydrate-containing agents. Such a work would be pivotal to improving the imaging efficacy and specificity in identifying anti-inflammatory/reparative macrophages, especially using MRI that has excellent spatial resolution and soft tissue contrast. Although single D-mannose-based imaging agents have been reported, as demonstrated herein (FIG. 2A), a single mannose moiety is unable to adequately differentiate M1 macrophages from M2 macrophages. This specificity issue in overcome herein by putting more mannose moieties on the imaging agents, given that two mannose units have a binding affinity over 100 times higher than that of D-mannose. Disclosed herein is the development of multi-mannose-based agents targeting CD206 and validation of their specificity and efficacy in detecting anti-inflammatory/reparative macrophages in relevant animal models of wound healing and glioma.

### Agent Development.

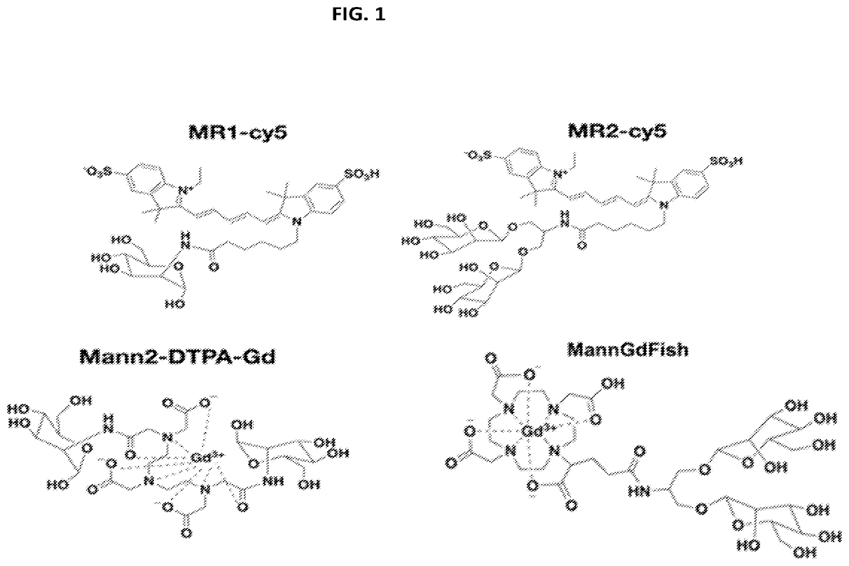

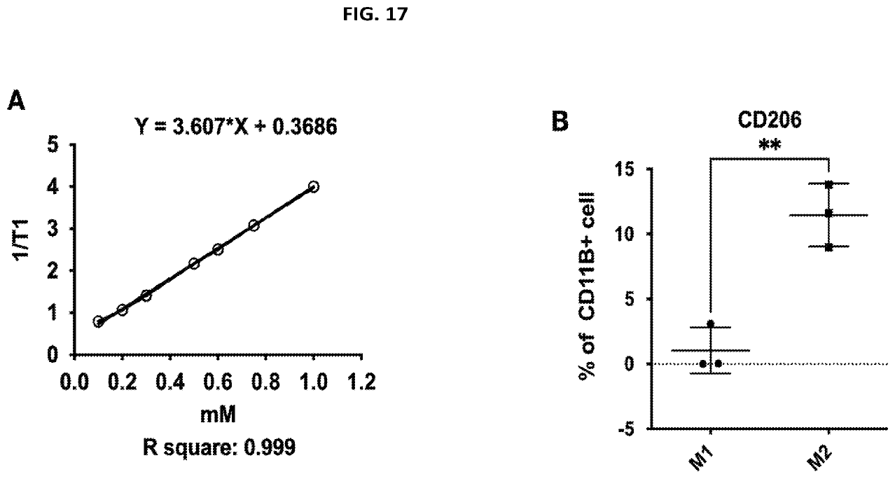

The MRI and fluorescent imaging agents, MR1-cy5, MR2-cy5, and Mann2-DTPA-Gd that contain one or two mannose moieties (FIG. 1) were designed to be specific to CD206 and anti-inflammatory/reparative macrophages. The detailed synthesis and characterization of these agents are described in the Examples. The relaxivity (r1) of Mann2-DTPA-Gd was 3.6 mM-1 s-1 in PBS (0.47 T, 40° C.) (FIG. 17A), and validation of differentiation of M1 and M2 macrophages is shown in FIG. 17B.

### In Vitro Validation of Specificity to the Mannose Receptor (CD206).

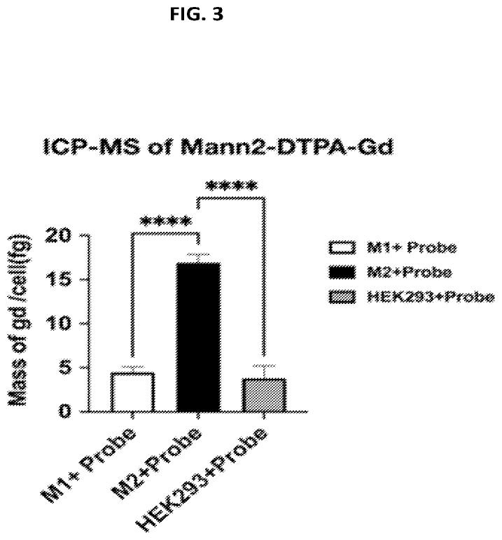

The specificity of MR1-cy5, MR2-cy5, and Mann2-DTPA-Gd to CD206 was validated in cellular assays using M1 and M2 macrophages differentiated from Raw 264.7 cells. The differentiation was confirmed by flow cytometry with a much higher percentage of CD206+ cells in M2 macrophages than that in M1 macrophages (see FIG. 17B). However, when incubated with MR1-cy5 containing only a single mannose moiety, fluorescent imaging showed similar signals from both M1 and M2 macrophages (FIG. 2A), demonstrating low specificity of MR1-cy5 to M2 macrophages. In contrast, fluorescent imaging from MR2-cy5, which has two mannose moieties in a clustered structure (FIG. 1), demonstrated a much higher signal in M2 macrophages compared to that in M1 macrophages and M2 macrophages incubated with MR1-cy5 (FIG. 2B). The MRI agent Mann2-DTPA-Gd was incubated with M1/M2 macrophages and human embryonic kidney cells (HEK293, a non-specific binding control). As expected, the amount of gadolinium in M2 macrophages was markedly higher than that in M1 macrophages and HEK293 cells (FIG. 3). Together these results confirmed the higher specificity of these agents containing two mannose units for M2 cells.

### MR Imaging of Mann2-DTPA-Gd in a Mouse Model of Cutaneous Wound Healing

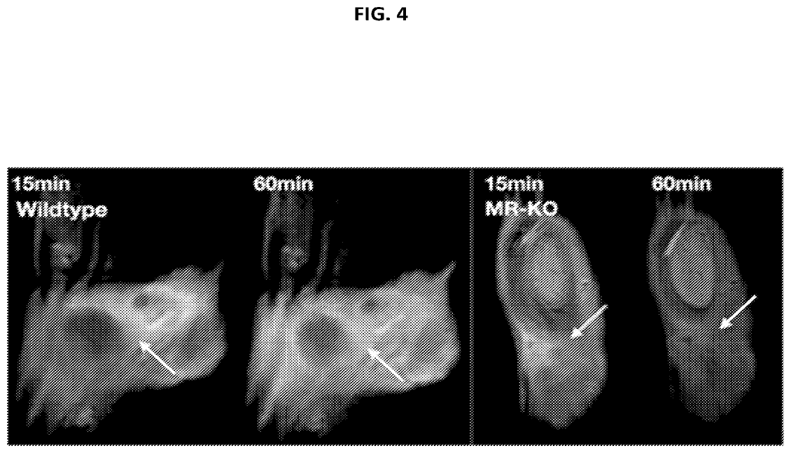

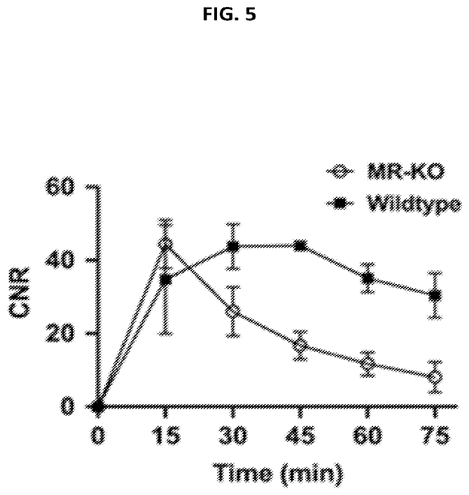

Normal wound healing is a highly regulated process in which activated macrophages function distinctively at different stages. In the early inflammatory stage post-injury (1-4 days), neutrophils and monocyte-derived macrophages are proinflammatory and produce inflammatory cytokines, proteases, and reactive oxygen species (ROS); on days 5-10 post-injury, inflammation starts to resolve and the reparative stage begins where anti-inflammatory and reparative macrophages become the most abundant cell type with the peak around day 7 and last throughout the reparative stage. The specificity and ability of Mann2-DTPA-Gd to track CD206⁺ macrophages in a mouse model of wound healing was evaluated on days 1, 4, and 7 since wound healing has well-defined inflammatory and reparative stages where proinflammatory and anti-inflammatory macrophages play distinctive roles. In wildtype mice on day 7 after wound induction, there was increased contrast enhancement at 60 min compared to 15 min after Mann2-DTPA-Gd administration (FIG. 4), with the contrast-to-noise ratio (CNR) increasing over time until 45 min post-injection prior to slowly decreasing (FIG. 5). On the other hand, in mannose receptor-knockout (MR-KO) mice, the signal intensity decreased over time after initially peaking at 15 min post-injection (FIG. 4 and FIG. 5), confirming the in vivo binding and specificity of Mann2-DTPA-Gd to the mannose receptor.

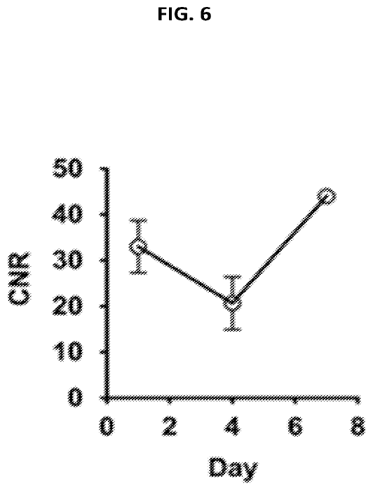

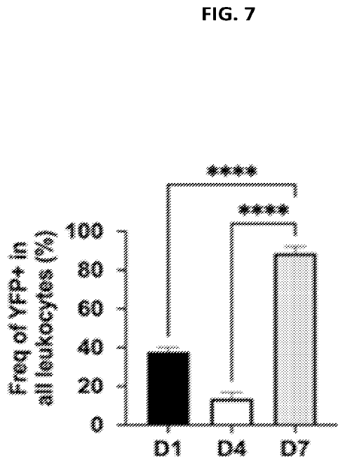

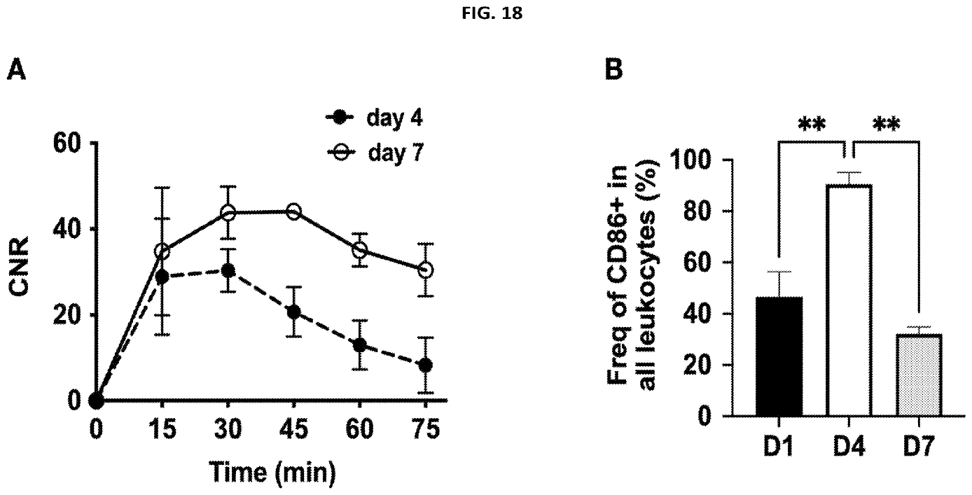

A longitudinal study was performed to track the changes in CD206⁺ macrophages in wound healing. Using the CNR at 60 min time point, it was found that some CD206+ cells were present on day 1 after wound induction which decreased on day 4 prior to increasing again on day 7 (FIG. 6). There was an approximately 2- to 3-fold higher CNR from 45-75 min post-injection on day 7 compared to that on day 4 (see FIG. 18A), demonstrating the evolution of CD206⁺ macrophages at different stages of healing. To validate the MRI data, a flow cytometric study was performed to differentiate proinflammatory and anti-inflammatory macrophages in the same model using transgenic YFP-labeled arginase I (YARG) mice on days 1, 4, and 7. Arginase 1 (Arg1) is another marker for M2-like macrophages. As expected, the flow cytometric data (FIG. 7) mirrored the results from Mann2-DTPA-Gd MRI (FIG. 6). To ensure that Mann2-DTPA-Gd imaging was not detecting proinflammatory macrophages, proinflammatory macrophages were also assessed by flow cytometry using CD86 as a marker, which showed an opposite trend (peaking on day 4) compared to what was detected by Mann2-DTPA-Gd (FIG. 18B). Taken together, these data demonstrated that MR imaging of Mann2-DTPA-Gd is capable of noninvasive mapping and tracking of the dynamic changes of CD206⁺ macrophages in wound healing.

MR Imaging of Experimental Glioma with Mann2-DTPA-Gd

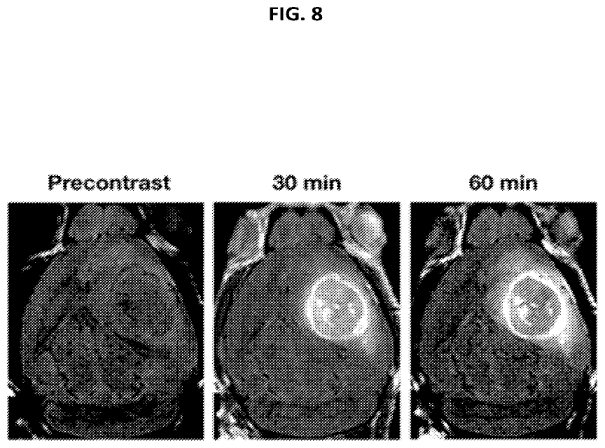

In contrast to the reparative role of CD206⁺ macrophages in wound healing, tumor-associated macrophages (TAMs) in cancer are the most abundant immune cells, and are tumorigenic and immunosuppressive in the tumor microenvironment. The TAMs facilitate tumor growth, immune evasion, and metastasis, and are associated with poor prognosis. Accordingly, it was next determined whether Mann2-DTPA-Gd can detect these cells in experimental glioma despite these cells serving different roles than in injury. Substantial contrast enhancement was observed after agent injection that increased over time (FIG. 8), revealing the presence of CD206⁺ TAMs in the tumor microenvironment. Interestingly, while there was mild increased signal with a few more intense foci within the tumor, the areas with the highest contrast enhancement formed a ring around the tumor, demonstrating that TAMs predominately surround the tumor. On the much-delayed images (60 min), there were additional areas of increased enhancement seen in the surrounding brain parenchyma. The data presented herein shows that CD206 MRI using Mann2-DTPA-Gd can detect CD206⁺ TAMs in a mouse model of glioma, and as expected, these TAMs were mainly distributed surrounding the tumor.

Similar to wound healing, these proinflammatory and anti-inflammatory/reparative stages also happen in myocardial infarction and acute kidney injury. Repolarization from an inflammatory phenotype to an anti-inflammatory/reparative phenotype would favor repair and recovery when M1-like macrophages/microglia exert inflammatory and damaging effects such as in diabetic wound healing and in many neurological diseases. Conversely, shifting from CD206⁺ TAMs to tumoricidal macrophages has become an important strategy to treat cancer and fibrosis. As in cancer, CD206⁺ macrophages are strongly associated with renal fibrosis both in human and experimental diseases. In renal fibrosis, a strategy to maintain or increase tissue repair while inhibiting the pro-fibrotic process of CD206⁺ macrophages represents a promising therapeutic strategy. Furthermore, recent studies have shown that CD206⁺ macrophages are associated with diabetes and adipose tissue lymphoid clusters in humans and correlate with adverse patient outcomes in human laryngeal squamous cell carcinoma, oral squamous cell carcinoma, and renal fibrosis and other renal diseases in patients. Given the specificity and efficacy of the imaging agents disclosed herein for CD206⁺ macrophages, they are anticipated be a valuable clinical tool. In some embodiments, the imaging agents disclosed herein provide a means to report healing and disease progression, and to monitor therapeutic effects in patients.

### Development of the Macrocyclic-Based MRI Agent MannGdFish

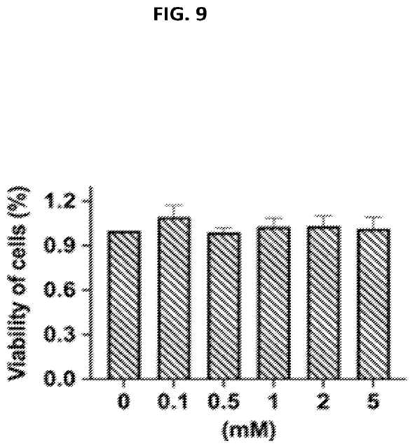

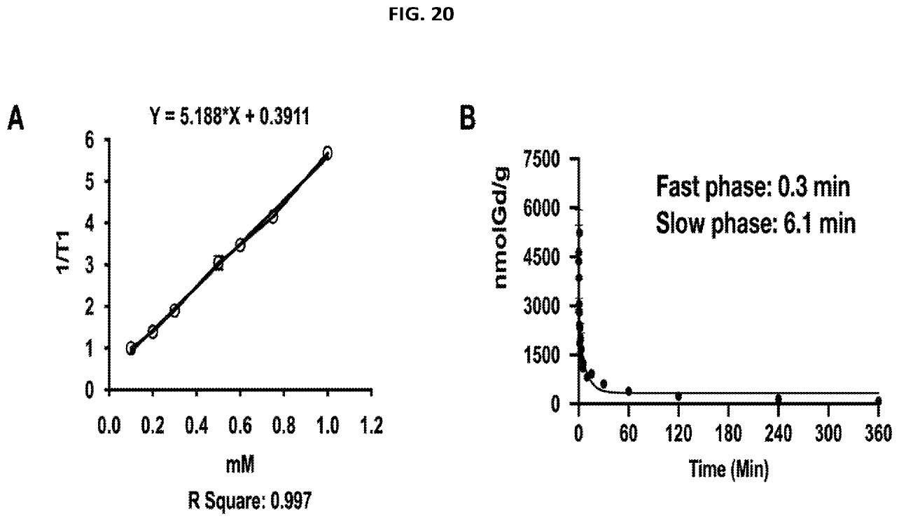

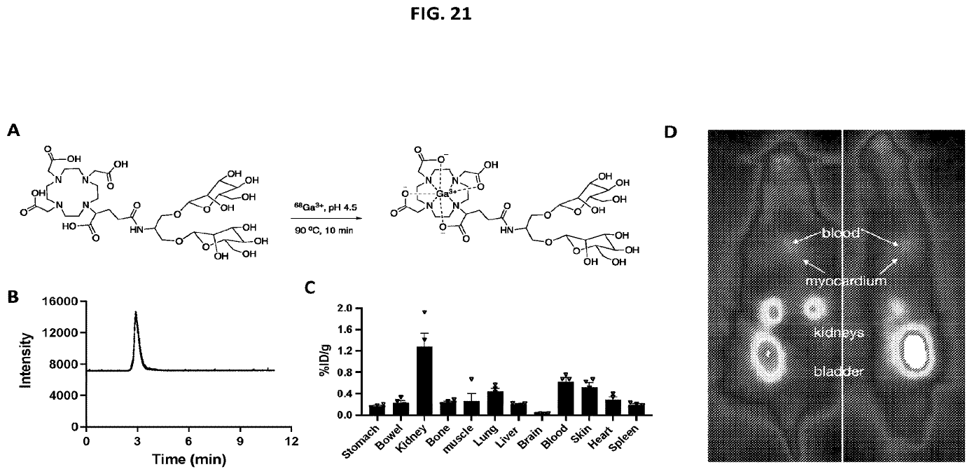

MR imaging of the prototype Mann2-DTPA-Gd proved it is feasible to use MRI to track CD206⁺ macrophages. However, since Mann2-DTPA-Gd contains a linear chelator, which has less Gd stability and thus undesirable for translation, a thermodynamically more stable macrocyclic-based agent was designed that resulted in MannGdFish (FIG. 1). The synthesis and characterization of MannGdFish is shown in Example 4. The relaxivity (r1) of MannGdFish is 5.2 mmol⁻¹ s⁻¹ (FIG. 20A), slightly higher than that of Mann2-DTPA-Gd (3.6 mmol-1 s-1). MannGdFish demonstrated no cytotoxic effect up to 5 mM (FIG. 9), a dose thousands of times higher than expected for first-pass concentration in the blood (μM), in MTT assays using RAW 264.7 cells.

### Biodistribution and Pharmacokinetics of MannGdFish.

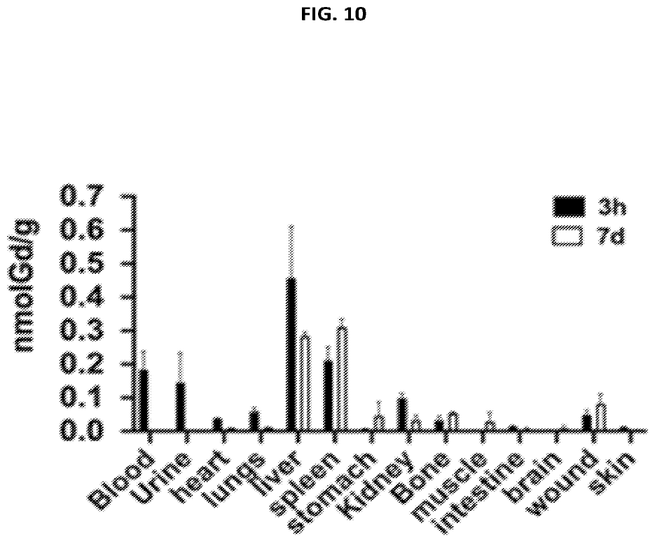

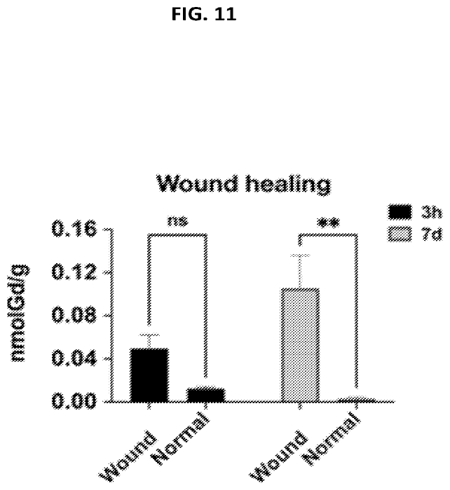

The biodistribution of MannGdFish in mice induced with a cutaneous wound was evaluated. ICP-MS overall showed very little accumulation of Gd or retention in the body (<0.5 nmol Gd per gram tissue), with liver and spleen being the major organs containing gadolinium at both 3 h and on day 7 followed by blood, urine, and kidneys (FIG. 10). As expected, the amount of gadolinium in the injured wound (24 h post-injury) at 3 h after MannGdFish administration was low and not significantly different compared to that in the normal skin, while the gadolinium content on day 7 was higher in the wound than that in the normal skin (FIG. 11), again highlighting the ability of MannGdFish to track CD206⁺ macrophages during wound healing. The blood half-life of MannGdFish for the fast phase was 0.3 min and 6.1 min for the slow phase using a two-phase exponential model (FIG. 20B).

### In Vivo Imaging of MannGdFish in Wound Healing

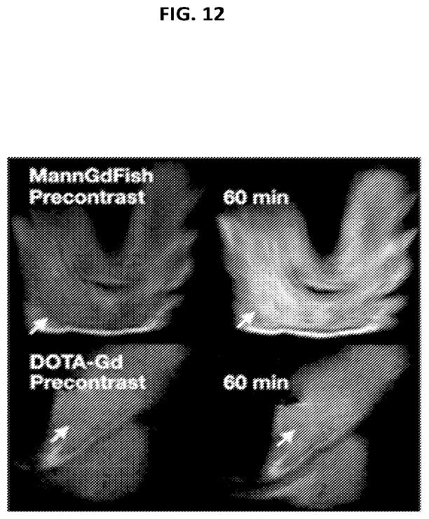

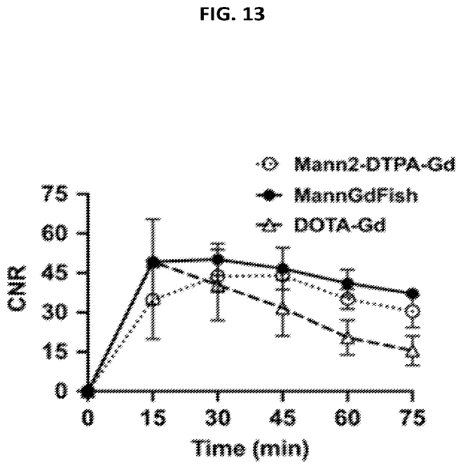

MR imaging of MannGdFish in wound healing on day 7 was performed and compared with that of Mann2-DTPA-Gd and DOTA-Gd (FIG. 12 and FIGS. 13A and 13B). The signal of MannGdFish at 60 min was much higher compared to that of DOTA-Gd (FIG. 12), consistent with binding and retention of MannGdFish to CD206+ cells. Unlike the slow decrease of CNR in MannGdFish and Mann2-DTPA-Gd after 45 min, the CNR of DOTA-Gd decreased rapidly after peaking at 15 min post-injection (FIG. 13A). The CNR of MannGdFish was slightly higher compared to that of Mann2-DTPA-Gd, consist with the higher r₁ of MannGdFish than that of Mann2-DTPA-Gd (3.6 vs. 5.2 mmol⁻¹ s⁻¹) and showed similar kinetic profile over 75 min as that of Mann2-DTPA-Gd. These results showed that MannGdFish has similar efficacy as Mann2-DTPA-Gd while exhibiting a significantly superior safety profile due to its macrocyclic chelating backbone, confirming it as a potential translational candidate for CD206⁺ macrophage MR imaging. Given the high sensitivity, specificity, stability, and favorable biodistribution and pharmacokinetics, MannGdFish is a promising translational candidate to monitor CD206⁺ macrophages in repair/regeneration and tumor in patients. In some embodiments, the imaging agents disclosed herein provide a means to monitor CD206⁺ macrophages in repair/regeneration and tumor in patients.

### Compounds of Formula (I)

Some embodiments provide a compound of Formula (I):

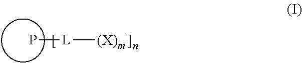

or a pharmaceutically acceptable salt thereof, wherein
P is a fluorescence imaging probe or a magnetic resonance imaging probe;
m is 1, 2, 3, 4, 5, 6, 7, 8, 9, 10, 11, or 12;
each X is independently X-1 or X-2:

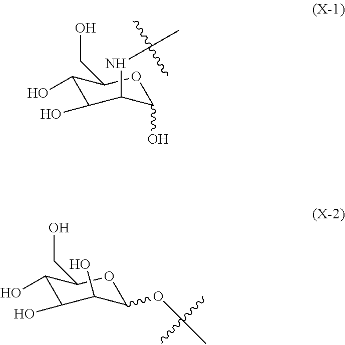 [SMILES: *NC1C(O)OC(CO)C(O)C1O ; *OC1OC(CO)C(O)C(O)C1O]

L is a C2-C20 alkylene; and
n is 1, 2, 3, 4, 5, or 6.

In some embodiments, L is a C2-C20 alkylene with 1-6 oxo groups and 1-6 nitrogen atoms. In some embodiments, L is a C2-C20 alkylene with 1-3 oxo groups and 1-3 nitrogen atoms. In some embodiments, L is a C2-C20 alkylene with 3-6 oxo groups and 3-6 nitrogen atoms. In some embodiments, L is a C2-C20 alkylene with 1 oxo group and 1 nitrogen atom. In some embodiments, L is a C2-C20 alkylene with 2 oxo groups and 2 nitrogen atoms. In some embodiments, L is a C2-C20 alkylene with 3 oxo groups and 3 nitrogen atoms. In some embodiments, L is a C2-C20 alkylene with 4 oxo groups and 4 nitrogen atoms. In some embodiments, L is a C2-C20 alkylene with 5 oxo groups and 5 nitrogen atoms. In some embodiments, L is a C2-C20 alkylene with 6 oxo groups and 6 nitrogen atoms.

In some embodiments, L is a C2-C20 alkylene with a phenyl group, 1-6 oxo groups, and 1-6 nitrogen atoms. In some embodiments, L is a C2-C20 alkylene with a phenyl group, 1-3 oxo groups, and 1-3 nitrogen atoms. In some embodiments, L is a C2-C20 alkylene with a phenyl group, 3-6 oxo groups, and 3-6 nitrogen atoms. In some embodiments, L is a C2-C20 alkylene with a phenyl group, 1 oxo group, and 1 nitrogen atom. In some embodiments, L is a C2-C20 alkylene with a phenyl group, 2 oxo groups, and 2 nitrogen atoms. In some embodiments, L is a C2-C20 alkylene with a phenyl group, 3 oxo groups, and 3 nitrogen atoms. In some embodiments, L is a C2-C20 alkylene with a phenyl group, 4 oxo groups, and 4 nitrogen atoms. In some embodiments, L is a C2-C20 alkylene with a phenyl group, 5 oxo groups, and 5 nitrogen atoms. In some embodiments, L is a C2-C20 alkylene with a phenyl group, 6 oxo groups, and 6 nitrogen atoms.

In some embodiments, L is a C2-C20 alkylene with 1-2 oxo groups and 0-2 nitrogen atoms. In some embodiments, L is a C2-C20 alkylene with one oxo group and 0-2 nitrogen atoms. In some embodiments, L is a C2-C20 alkylene with one oxo group and 0-1 nitrogen atoms. In some embodiments, L is a C2-C20 alkylene with one oxo group. In some embodiments, L is a C2-C20 alkylene with one oxo group and 1 nitrogen atom. In some embodiments, L is a C2-C20 alkylene with one oxo group and 2 nitrogen atoms. In some embodiments, L is a C2-C20 alkylene with two oxo groups and 0-1 nitrogen atoms. In some embodiments, L is a C2-C20 alkylene with two oxo groups. In some embodiments, L is a C2-C20 alkylene with two oxo groups and 1 nitrogen atom. In some embodiments, L is a C2-C20 alkylene with two oxo groups and 2 nitrogen atoms.

In some embodiments, L is a C2-C20 alkylene with a phenyl group, 1-2 oxo groups, and 0-2 nitrogen atoms. In some embodiments, L is a C2-C20 alkylene with a phenyl group, one oxo group, and 0-2 nitrogen atoms. In some embodiments, L is a C2-C20 alkylene with a phenyl group, one oxo group, and 0-1 nitrogen atoms. In some embodiments, L is a C2-C20 alkylene with a phenyl group and one oxo group. In some embodiments, L is a C2-C20 alkylene with a phenyl group, one oxo group, and 1 nitrogen atom. In some embodiments, L is a C2-C20 alkylene with a phenyl group, one oxo group, and 2 nitrogen atoms. In some embodiments, L is a C2-C20 alkylene with a phenyl group, two oxo groups, and 0-1 nitrogen atoms. In some embodiments, L is a C2-C20 alkylene with a phenyl group and two oxo groups. In some embodiments, L is a C2-C20 alkylene with a phenyl group, two oxo groups, and 1 nitrogen atom. In some embodiments, L is a C2-C20 alkylene with a phenyl group, two oxo groups, and 2 nitrogen atoms.

In some embodiments, L is a C2-C12 alkylene with 1-2 oxo groups and 0-2 nitrogen atoms. In some embodiments, L is a C2-C12 alkylene with one oxo group and 0-2 nitrogen atoms. In some embodiments, L is a C2-C12 alkylene with one oxo group and 0-1 nitrogen atoms. In some embodiments, L is a C2-C12 alkylene with one oxo group. In some embodiments, L is a C2-C12 alkylene with one oxo group and 1 nitrogen atom. In some embodiments, L is a C2-C12 alkylene with one oxo group and 2 nitrogen atoms. In some embodiments, L is a C2-C12 alkylene with two oxo groups and 0-1 nitrogen atoms. In some embodiments, L is a C2-C12 alkylene with two oxo groups. In some embodiments, L is a C2-C12 alkylene with two oxo groups and 1 nitrogen atom. In some embodiments, L is a C2-C12 alkylene with two oxo groups and 2 nitrogen atoms.

In some embodiments, L is a C2-C10 alkylene with 1-2 oxo groups and 0-2 nitrogen atoms. In some embodiments, L is a C2-C10 alkylene with one oxo group and 0-2 nitrogen atoms. In some embodiments, L is a C2-C10 alkylene with one oxo group and 0-1 nitrogen atoms. In some embodiments, L is a C2-C10 alkylene with one oxo group. In some embodiments, L is a C2-C10 alkylene with one oxo group and 1 nitrogen atom. In some embodiments, L is a C2-C10 alkylene with one oxo group and 2 nitrogen atoms. In some embodiments, L is a C2-C10 alkylene with two oxo groups and 0-1 nitrogen atoms. In some embodiments, L is a C2-C10 alkylene with two oxo groups. In some embodiments, L is a C2-C10 alkylene with two oxo groups and 1 nitrogen atom. In some embodiments, L is a C2-C10 alkylene with two oxo groups and 2 nitrogen atoms.

In some embodiments, L is a C2-C8 alkylene with 1-2 oxo groups and 0-2 nitrogen atoms. In some embodiments, L is a C2-C8 alkylene with one oxo group and 0-2 nitrogen atoms. In some embodiments, L is a C2-C8 alkylene with one oxo group and 0-1 nitrogen atoms. In some embodiments, L is a C2-C8 alkylene with one oxo group. In some embodiments, L is a C2-C8 alkylene with one oxo group and 1 nitrogen atom. In some embodiments, L is a C2-C8 alkylene with one oxo group and 2 nitrogen atoms. In some embodiments, L is a C2-C8 alkylene with two oxo groups and 0-1 nitrogen atoms. In some embodiments, L is a C2-C8 alkylene with two oxo groups. In some embodiments, L is a C2-C8 alkylene with two oxo groups and 1 nitrogen atom. In some embodiments, L is a C2-C8 alkylene with two oxo groups and 2 nitrogen atoms.

In some embodiments, L comprises one or more polyethylene glycol units.

In some embodiments, L comprises one or more amide groups (—NH(C═O)— or —(C═O)NH—).

In some embodiments, L is

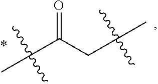 [SMILES: *CC(*)=O ; ","]

wherein * indicates the point of attachment to X.

In some embodiments, L is

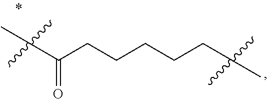 [SMILES: *CCCCCC(*)=O]

wherein * indicates the point of attachment to X.

In some embodiments, L is

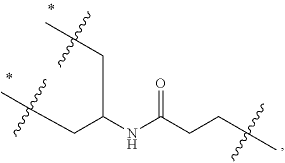 [SMILES: *CCC(=O)NC(C*)C*]

wherein * indicates the point of attachment to X.

In some embodiments, L is

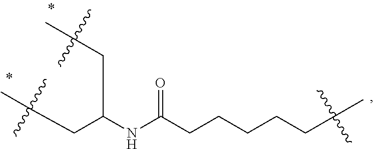 [SMILES: *CCCCCC(=O)NC(C*)C*]

wherein * indicates the point of attachment to X.

In some embodiments, X is X-1.

In some embodiments, X is X-2.

In some embodiments, each X is X-1. In some embodiments, each X is X-2. In some embodiments, when m≥2, one or more X is X-1 and one or more is X-2. In some embodiments, each X is the same. In some embodiments, each X is different.

In some embodiments, when m≥2, L is connected to multiple independently selected X groups, as described herein.

In some embodiments, L is

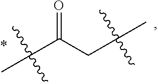 [SMILES: *CC(*)=O ; ","]

wherein * indicates the point of attachment to X-1.

In some embodiments, L is

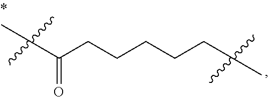 [SMILES: *CCCCCC(*)=O]

wherein * indicates the point of attachment to X-1.

In some embodiments, L is

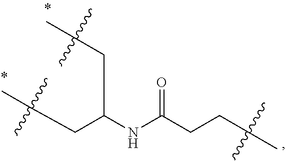 [SMILES: *CCC(=O)NC(C*)C*]

wherein * indicates the point of attachment to X-1.

In some embodiments, L is

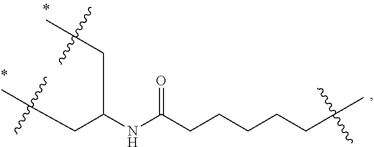 [SMILES: *CCCCCC(=O)NC(C*)C*]

wherein * indicates the point of attachment to X-1.

In some embodiments, L is

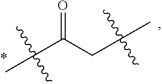 [SMILES: *CC(*)=O ; ","]

wherein * indicates the point of attachment to X-2.

In some embodiments, L is

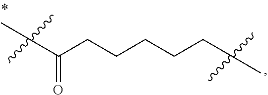 [SMILES: *CCCCCC(*)=O]

wherein * indicates the point of attachment to X-2.

In some embodiments, L is

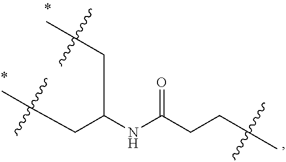 [SMILES: *CCC(=O)NC(C*)C*]

wherein * indicates the point of attachment to X-2.

In some embodiments, L is

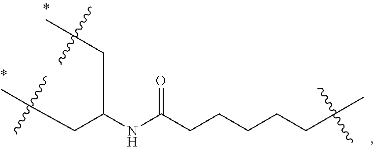 [SMILES: *CCCCCC(=O)NC(C*)C*]

wherein * indicates the point of attachment to X-2.

In some embodiments, L is

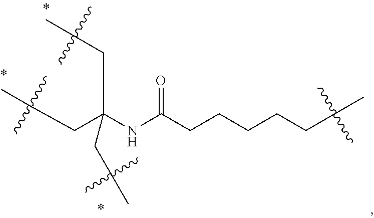 [SMILES: *CCCCCC(=O)NC(C*)(C*)C*]

wherein * indicates the point of attachment to X-2.

In some embodiments, L is

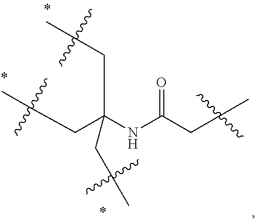 [SMILES: *CC(=O)NC(C*)(C*)C*]

wherein * indicates the point of attachment to X-2.

In some embodiments, L is

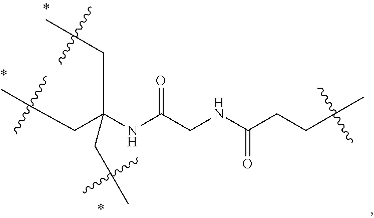 [SMILES: *CCC(=O)NCC(=O)NC(C*)(C*)C*]

wherein * indicates the point of attachment to X-2.

In some embodiments, L is

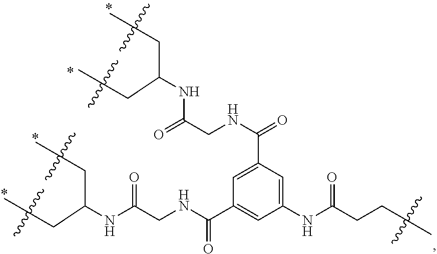 [SMILES: *CCC(=O)Nc1cc(C(=O)NCC(=O)NC(C*)C*)cc(C(=O)NCC(=O)NC(C*)C*)c1]

wherein * indicates the point of attachment to X-2.

In some embodiments, L is

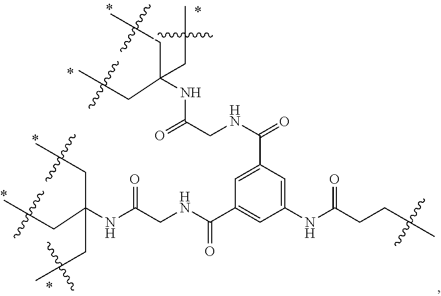 [SMILES: *CCC(=O)Nc1cc(C(=O)NCC(=O)NC(C*)(C*)C*)cc(C(=O)NCC(=O)NC(C*)(C*)C*)c1]

wherein * indicates the point of attachment to X-2.

In some embodiments, L is

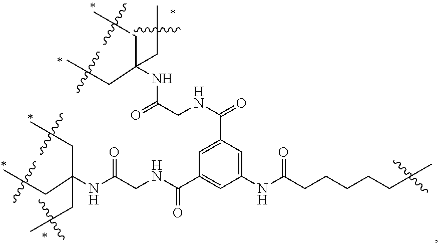 [SMILES: *CCCCCC(=O)Nc1cc(C(=O)NCC(=O)NC(C*)(C*)C*)cc(C(=O)NCC(=O)NC(C*)(C*)C*)c1]

wherein * indicates the point of attachment to X-2.

In some embodiments, L is

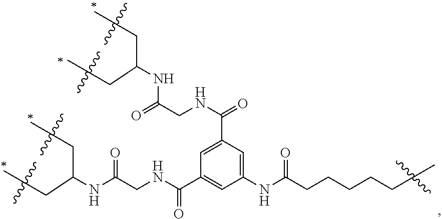 [SMILES: *CCCCCC(=O)Nc1cc(C(=O)NCC(=O)NC(C*)C*)cc(C(=O)NCC(=O)NC(C*)C*)c1]

wherein * indicates the point of attachment to X-2.

In some embodiments, P is a fluorescence imaging probe.

In some embodiments, P is cy3 or cy5. In some embodiments, P is cy3. In some embodiments, P is cy5.

In some embodiments, P is

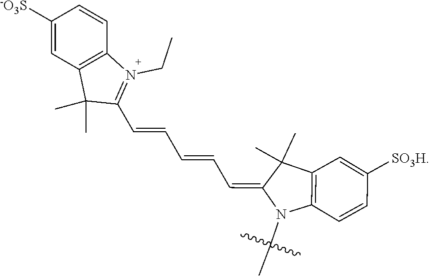

In some embodiments, P is a magnetic resonance imaging probe.

In some embodiments, P is

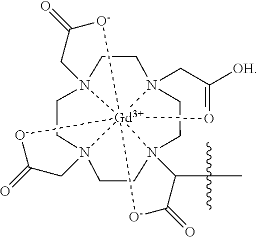

In some embodiments, P is

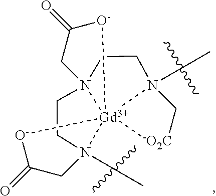

In some embodiments, P is

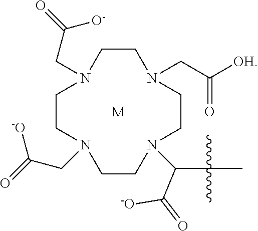

In some embodiments, P is

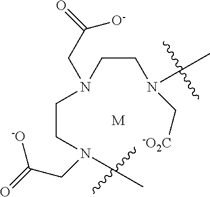

In some embodiments, P is

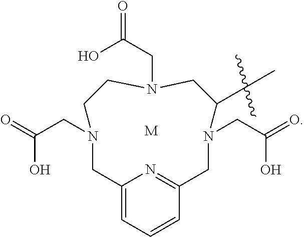

In some embodiments, P is

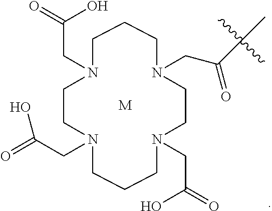

In some embodiments, P is

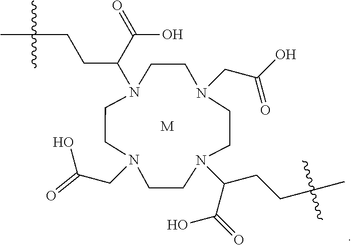

In some embodiments, P is

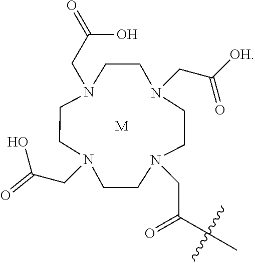

In some embodiments, the radioisotope, M, is selected from the group consisting of radioisotopes of Ga, In, Mn, and Cu.

In some embodiments, the radioisotope, M, is selected from the group consisting of ⁶⁸Ga, ¹¹¹In, ⁵²Mn, and ⁶⁴Cu.

In some embodiments, the radioisotope, M, is ⁶⁸Ga.

In some embodiments, the radioisotope, M, is ¹¹¹In.

In some embodiments, the radioisotope, M, is ⁵²Mn.

In some embodiments, the radioisotope, M, is ⁶⁴Cu.

In some embodiments, n is 1 or 2. In some embodiments, n is 1. In some

embodiments, n is 2. In some embodiments, n is 3 or 4. In some embodiments, n is 3. In some embodiments, n is 4. In some embodiments, n is 5 or 6. In some embodiments, n is 5. In some embodiments, n is 6.

In some embodiments, m is 1, 2, 3, 4, 5, or 6. In some embodiments, m is 1, 2, or 3. In some embodiments, m is 4, 5, or 6. In some embodiments, m is 7, 8, 9, 10, 11, or 12. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12.

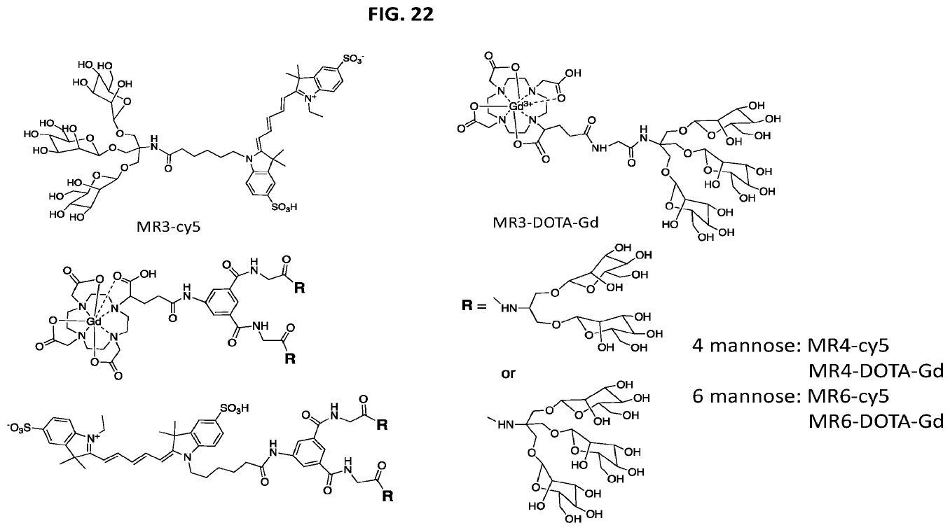

In some embodiments, the compound of Formula (I) is selected from the compounds in FIG. 1, FIG. 22, or a pharmaceutically acceptable salt thereof. In some embodiments, the compound of Formula (I) is selected from the group consisting of:

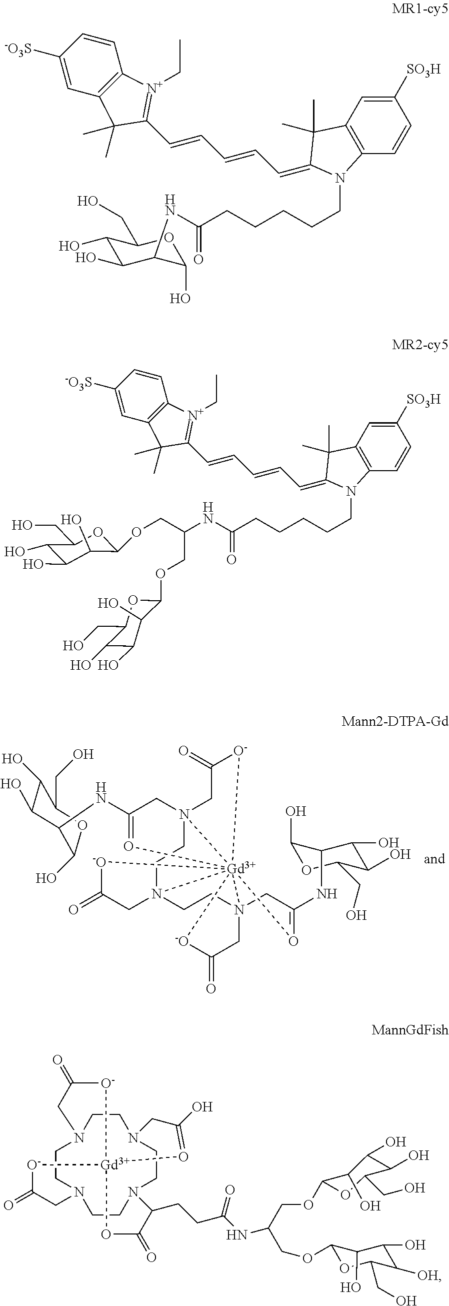

or a pharmaceutically acceptable salt of any of the foregoing.

In some embodiments, the compound of Formula (I) is selected from the group consisting of:

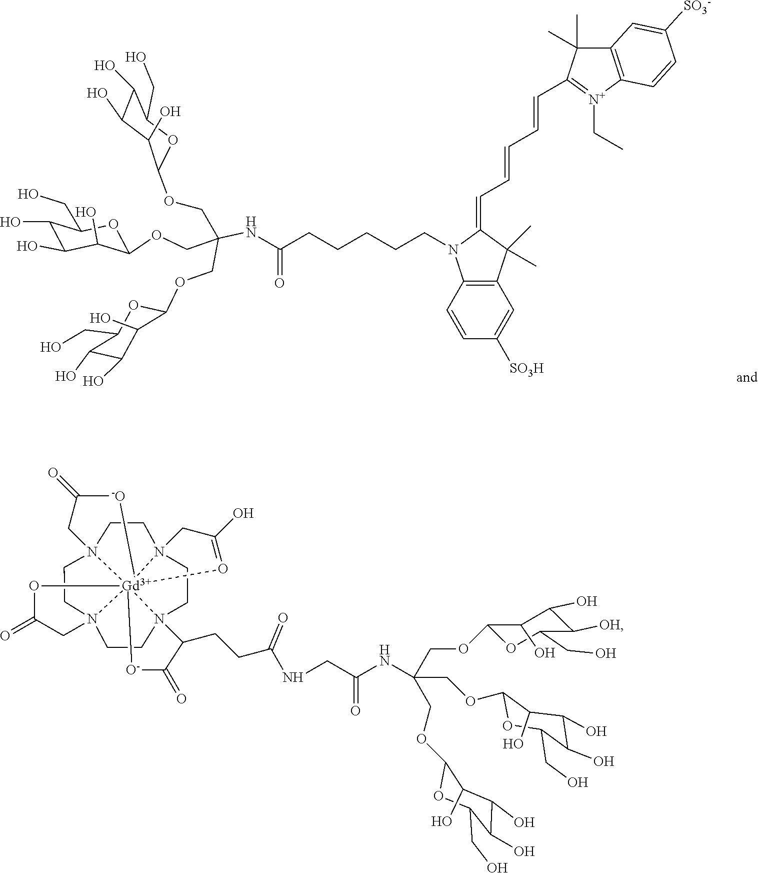

or a pharmaceutically acceptable salt of any of the foregoing.

In some embodiments, the compound of Formula (I) is selected from the group consisting of:

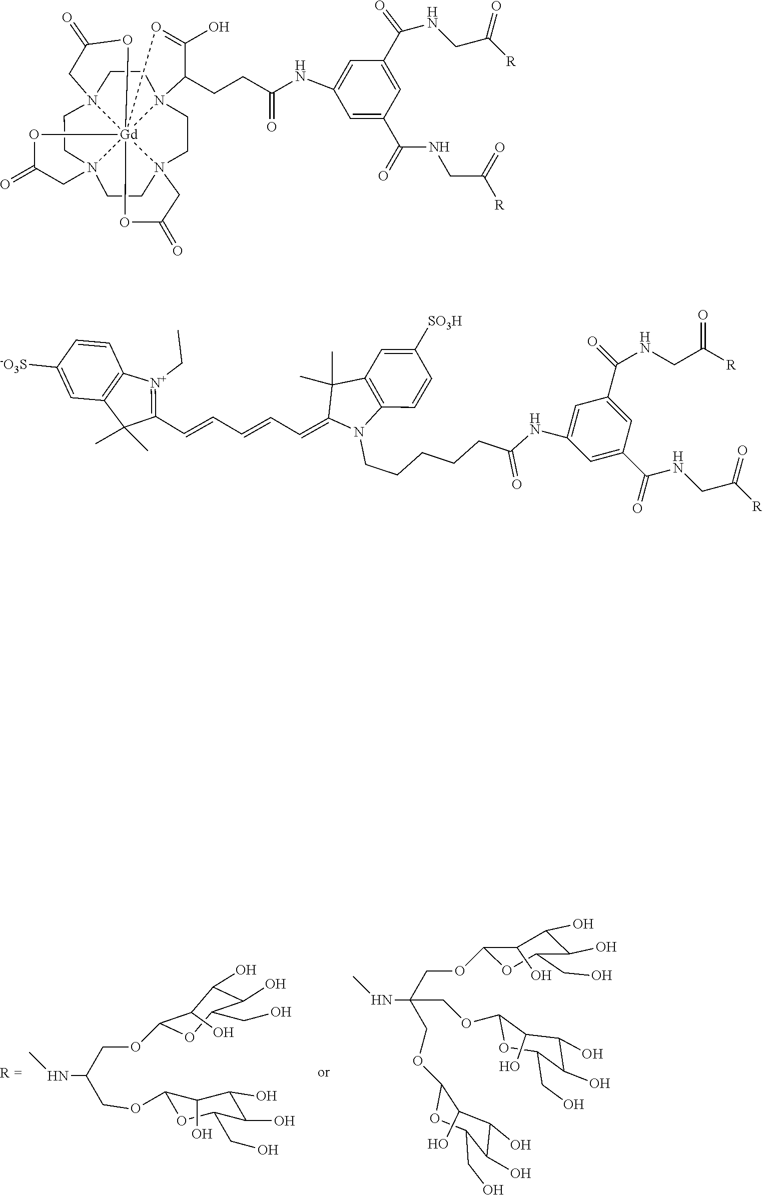

or a pharmaceutically acceptable salt of any of the foregoing.

In some embodiments, the compound of Formula (I), or a pharmaceutically acceptable salt thereof, is MR1-cy5, or a pharmaceutically acceptable salt thereof.

In some embodiments, the compound of Formula (I), or a pharmaceutically acceptable salt thereof, is MR2-cy5, or a pharmaceutically acceptable salt thereof.

In some embodiments, the compound of Formula (I), or a pharmaceutically acceptable salt thereof, is Mann2-DTPA-Gd, or a pharmaceutically acceptable salt thereof.

In some embodiments, the compound of Formula (I), or a pharmaceutically acceptable salt thereof, is MannGdFish, or a pharmaceutically acceptable salt thereof.

### Methods of Use

Some embodiments provide a method of detecting M2-like macrophages in vitro, the method comprising:
(i) contacting a compound of Formula (I) with cells or tissues;
(ii) waiting a time to allow the compound to accumulate in the cells or tissues; and
(iii) acquiring an image of the cells or tissues.

In some embodiments, the cells are isolated from tissues. In some embodiments, the tissues are animal tissues. In some embodiments, the tissues are from a human. In some embodiments, the cells comprise a biopsy sample.

In some embodiments, the cells or tissues are selected cells or tissues from an artery, a vein, a lymph node, a lung, a liver, a kidney, a skin, a brain, an eye, a bone, an intestine, a gallbladder, a pancreas, a trachea, a bladder, a bowel, a biliary tract, an adrenal gland, a uterus, an ovary, a spleen, a cartilage, a muscle, a heart, a cartilage, an epithelium, a tendon, and a ligament. In some embodiments, the cells or tissues are cells or tissues from an organ. In some embodiments, the cells or tissues are cells and tissues from connective tissue.

Some embodiments provide a method of monitoring M2-like macrophages in an organ or a tissue of a subject, the method comprising:
(i) administering to the subject an effective amount of a compound of Formula (I), or a pharmaceutically acceptable salt thereof, or a pharmaceutical composition as described herein;
(ii) waiting a time sufficient to allow the compound to accumulate in the organ or a tissue to be imaged; and
(iii) acquiring an image of the organ or the tissue of the subject,
wherein observing the image attributable to the compound of Formula (I) is indicative of M2-like macrophages in an organ or tissue.

In some embodiments, the organ or the tissue comprises an area affected by an injury, cardiovascular disease, inflammation, a neurodegenerative disease, or cancer, or a combination of any of the foregoing. In some embodiments, the organ or the tissue comprises an area affected by an injury, cardiovascular disease, inflammation, a neurodegenerative disease, or cancer.

Some embodiments provide a method of monitoring treatment of a disease or condition in a subject, the method comprising:
(i) administering to the subject an effective amount of a compound of Formula (I), or a pharmaceutically acceptable salt thereof, or a pharmaceutical composition as described herein;
(ii) waiting a time sufficient to allow the compound to accumulate in an organ or tissue of the subject affected by the disease or condition;
(iii) acquiring a first image of the organ or the tissue of the subject;
(iv) administering to the subject a therapeutic agent in an effective amount to treat the disease or condition;
(v) administering to the subject an effective amount of a compound of Formula (I), or a pharmaceutically acceptable salt thereof, or a pharmaceutical composition as described herein;
(vi) waiting a time sufficient to allow the compound to accumulate in the organ or tissue of the subject affected by the disease or condition;
(vii) acquiring a second image of the organ or the tissue of the subject; and
(viii) comparing the first and the second images, wherein observing a difference between the first and the second images attributable to the compound of Formula (I) is indicative of progression of treatment of the disease or condition.

Some embodiments provide a method of diagnosing a disease or condition in a subject, the method comprising:
(i) administering to the subject an effective amount of a compound of Formula (I), or a pharmaceutically acceptable salt thereof, or a pharmaceutical composition as described herein;
(ii) waiting a time sufficient to allow the compound to accumulate in an organ or a tissue of the subject affected by the disease or condition; and
(iii) acquiring an image of the organ or the tissue of the subject, wherein observing the image attributable to the compound of Formula (I) is indicative of the disease or condition.

In some embodiments, the organ or the tissue comprises an area affected by an injury. In some embodiments, the organ or the tissue comprises an area affected by cardiovascular disease. In some embodiments, the organ or the tissue comprises an area affected by inflammation. In some embodiments, the organ or the tissue comprises an area affected by a neurodegenerative disease. In some embodiments, the organ or the tissue comprises an area affected by cancer.

In some embodiments, the organ or the tissue is selected from an artery, a vein, a lymph node, a lung, a liver, a kidney, a skin, a brain, an eye, a bone, an intestine, a gallbladder, a pancreas, a trachea, a bladder, a bowel, a biliary tract, an adrenal gland, a uterus, an ovary, a spleen, a cartilage, a muscle, a heart, a cartilage, an epithelium, a tendon, and a ligament.

In some embodiments, the disease or condition is an injury, cardiovascular disease, inflammation, a neurodegenerative disease, or cancer, or a combination of any of the foregoing. In some embodiments, the disease or condition is an injury, cardiovascular disease, inflammation, a neurodegenerative disease, or cancer.

In some embodiments, the disease or condition is an injury. In some embodiments, the disease or condition is cardiovascular disease. In some embodiments, the disease or condition is inflammation. In some embodiments, the disease or condition is an a neurodegenerative disease. In some embodiments, the disease or condition is cancer.

Some embodiments provide a method of intraoperative imaging in a subject, the method comprising:
(i) administering to the subject an effective amount of a compound of Formula (I), or a pharmaceutically acceptable salt thereof, or a pharmaceutical composition as described herein;
(ii) waiting a time sufficient to allow the compound to accumulate in an organ or a tissue of the subject affected by the disease or condition; and
(iii) acquiring an image of the organ or the tissue of the subject, wherein observing the image attributable to the compound of Formula (I) defines margins.

In some embodiments, the disease or condition comprises a tumor or a lesion. In some embodiments, the disease or condition is a tumor or a lesion. In some embodiments, the disease or condition is a tumor. In some embodiments, the disease or condition is a lesion.

In some embodiments, the image comprises a fluorescence image. In some embodiments, the image is a fluorescence image.

In some embodiments, the image comprises a magnetic resonance image. In some embodiments, the image is a magnetic resonance image.

Some embodiments provide a method of detecting M2-like macrophages in vitro, the method comprising:
(i) contacting a compound selected from MR1-cy5, MR2-cy5, Mann2DTPA, and MannGDFish, with cells or tissues;
(ii) waiting a time to allow the compound to accumulate in the cells or tissues; and
(iii) acquiring an image of the cells or tissues.

Some embodiments provide a method of monitoring M2-like macrophages in an organ or a tissue of a subject, the method comprising:
(i) administering to the subject an effective amount of a compound selected from MR1-cy5, MR2-cy5, Mann2DTPA, and MannGDFish, or a pharmaceutically acceptable salt of any of the foregoing, or a pharmaceutical composition as described herein;
(ii) waiting a time sufficient to allow the compound to accumulate in the organ or a tissue to be imaged; and
(iii) acquiring an image of the organ or the tissue of the subject,
wherein observing the image attributable to the compound is indicative of M2-like macrophages in an organ or tissue.

Some embodiments provide a method of monitoring treatment of a disease or condition in a subject, the method comprising:
(i) administering to the subject an effective amount of a compound selected from MR1-cy5, MR2-cy5, Mann2DTPA, and MannGDFish, or a pharmaceutically acceptable salt of any of the foregoing, or a pharmaceutical composition as described herein;
(ii) waiting a time sufficient to allow the compound to accumulate in an organ or tissue of the subject affected by the disease or condition;
(iii) acquiring a first image of the organ or the tissue of the subject;
(iv) administering to the subject a therapeutic agent in an effective amount to treat the disease or condition;
(v) administering to the subject an effective amount of a compound selected from MR1-cy5, MR2-cy5, Mann2DTPA, and MannGDFish, or a pharmaceutically acceptable salt of any of the foregoing, or a pharmaceutical composition as described herein;
(vi) waiting a time sufficient to allow the compound to accumulate in the organ or tissue of the subject affected by the disease or condition;
(vii) acquiring a second image of the organ or the tissue of the subject; and
(viii) comparing the first and the second images,
wherein observing a difference between the first and the second images attributable to the compound is indicative of progression of treatment of the disease or condition.

Some embodiments provide a method of diagnosing a disease or condition in a subject, the method comprising:
(i) administering to the subject an effective amount of a compound selected from MR1-cy5, MR2-cy5, Mann2DTPA, and MannGDFish, or a pharmaceutically acceptable salt of any of the foregoing, or a pharmaceutical composition as described herein;
(ii) waiting a time sufficient to allow the compound to accumulate in an organ or a tissue of the subject affected by the disease or condition; and
(iii) acquiring an image of the organ or the tissue of the subject,
   wherein observing the image attributable to the compound is indicative of the disease or condition.

Some embodiments provide a method of intraoperative imaging in a subject, the method comprising:
(i) administering to the subject an effective amount of a compound selected from MR1-cy5, MR2-cy5, Mann2DTPA, and MannGDFish, or a pharmaceutically acceptable salt of any of the foregoing, or a pharmaceutical composition as described herein;
(ii) waiting a time sufficient to allow the compound to accumulate in an organ or a tissue of the subject affected by the disease or condition; and
(iii) acquiring an image of the organ or the tissue of the subject,
   wherein observing the image attributable to the compound defines margins.

### Compositions and Routes of Administration

The present application also provides pharmaceutical compositions comprising an effective amount of a compound of the present disclosure (e.g., MannGdFish, or a pharmaceutically acceptable salt thereof) disclosed herein, or a pharmaceutically acceptable salt thereof; and a pharmaceutically acceptable carrier.

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

In the pharmaceutical compositions of the present application, a compound of the present disclosure (e.g., MannGdFish or Mann2-DTPA-Gd) is present in an effective amount (e.g., an imaging effective amount).

### Examples

### Materials and Methods

All chemicals were obtained from Sigma Chemical Co. unless otherwise stated. D-mannosamine hydrochloride was purchased from Biosynth Carbosynth (United 29ingdom), DOTA-GA anhydride from CheMatech (Dijon, France), Z-Ser-OH from Ambeed (Illinois, USA) and 1,2,3,4,6-penta-O-benzoyl-α-D-mannopyranose from BOC Science (New York, USA). ¹H-NMR and ¹³C-NMR were recorded with a JEOL 11.7 T NMR system equipped with a 5 mm broadband probe. Flash chromatography was performed with Combiflash (Telydyne ISCO CombiFlash, CA) with UV detection at 220 and 254 nm. High-resolution mass spectrometry was performed with Thermo Scientific™ Q-Exactive Plus Ultimate 3000 HPLC flow injection analysis. Inductively coupled plasma mass spectrometry (ICP-MS) was conducted on an Agilent 8800-QQQ system. Flow cytometry data were acquired with LSRII flow cytometer (BD Bioscience). All animal experiments were carried out in compliance with the National Institutes of Health's ‘Guide for the Care and Use of Laboratory Animals’ and were approved by and in compliance with the Institutional Animal Care and Use Committee at Massachusetts General Hospital.

Statistical analysis. All numeric data were first analyzed for normality using the Shapiro-Wilk normality test with significance to determine the appropriate parametric or nonparametric test to use. One-way ANOVA and two-way ANOVA were used for the data analyses. All statistical analyses were performed with GraphPad Prism version 9 (GraphPad Software, La Jolla California) and data are shown as mean±SEM. P-values <0.05 were considered significantly different.

### CHEMICAL EXAMPLES

MR1-cy5 was synthesized by coupling D-mannosamine with cy5-NHS-ester (Scheme 1). A prototype MRI agent, Mann2-DTPA-Gd was synthesized by coupling of D-mannosamine with DTPA anhydride under basic conditions followed by chelating with GdCl3 (Scheme 2). Intermediate 8 obtained from coupling of compound 5 and 6 followed by hydrogenation was coupled with cy5-NHS ester followed by deprotection to provide MR2-cy5 (Scheme 3). Synthesis of MannGdFish: intermediate 8 was reacted with compound 10 (DOTA-GA anhydride) to give compound 11, followed by deprotection with sodium methoxide and chelation with GdCl3 to provide the final product MannGdFish (Scheme 4).

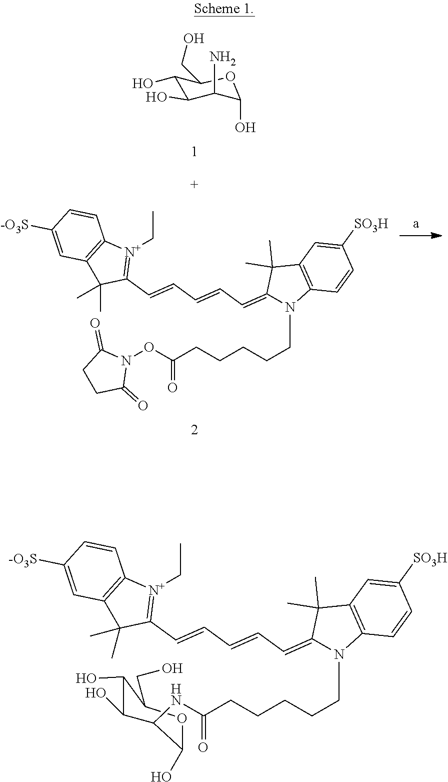

### Preparation of MR1-cy5. Step a Net₃, DMSO, rt, 35%.

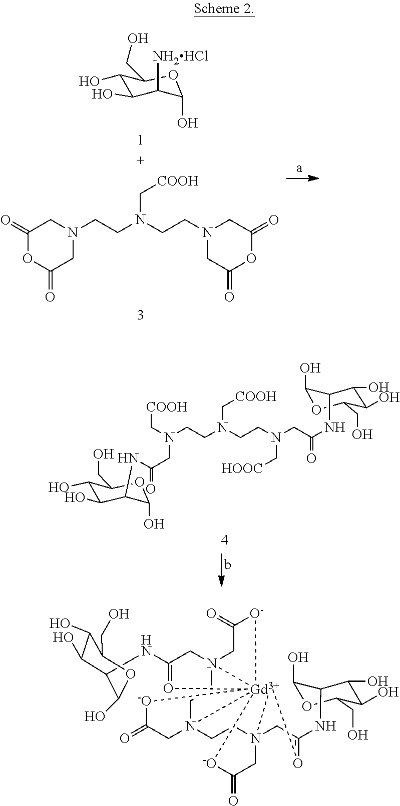

Preparation of Mann2-DTPA-Gd. Steps: a) Net₃, DMSO, rt; b) GdCl₃, sodium citrate buffer, pH 5.5. The total yield for two step 32%.

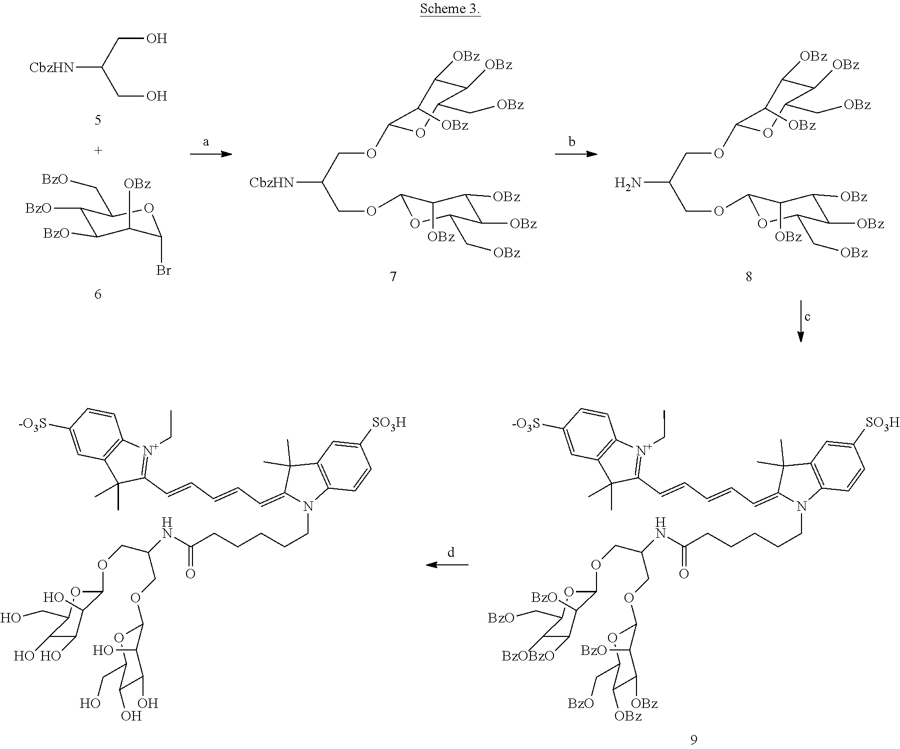

Preparation of MR2-cy5. Steps: a) AgOTf, 4 Å molecular sieves, rt, 65%; b) H₂, Pd/C, MeOH, rt, quantitative; c) Cy5-NHS ester, Net₃, DMSO; d) NaOMe/MeOH, 31% for the last two steps.

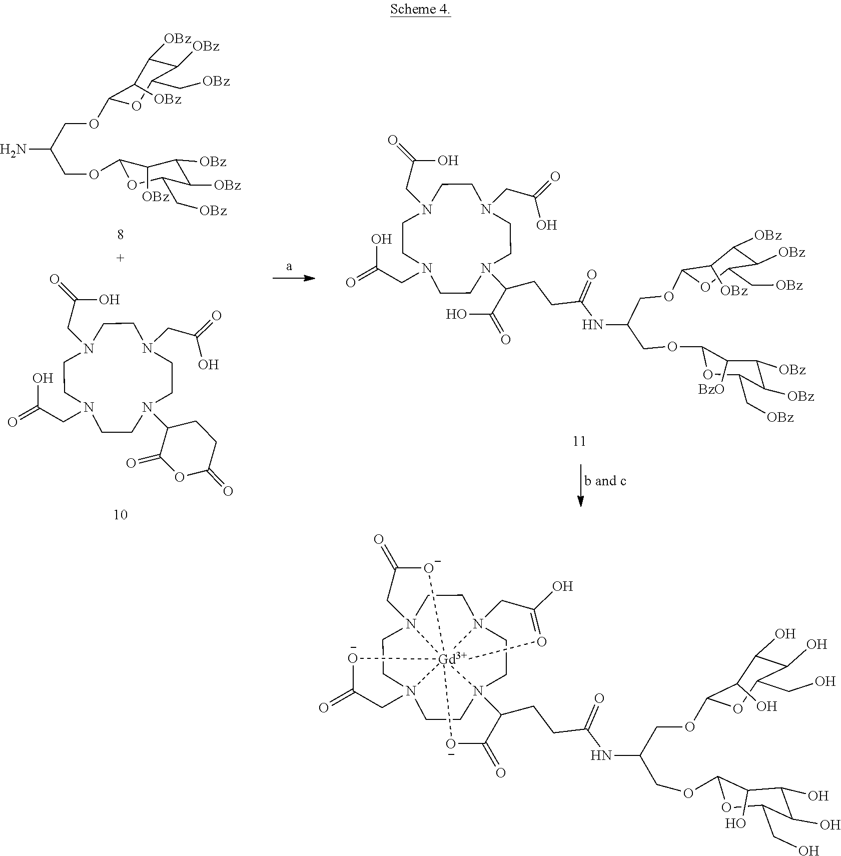

Preparation of MannGdFish. Steps: a) DIPEA, DMSO, 70° C., overnight, 71%; b) NaOMe/MeOH, rt; c) GdCl₃, pH 5.5, sodium acetate buffer, 45% for the last two steps.

### Example 1. Preparation of MR1-cy5

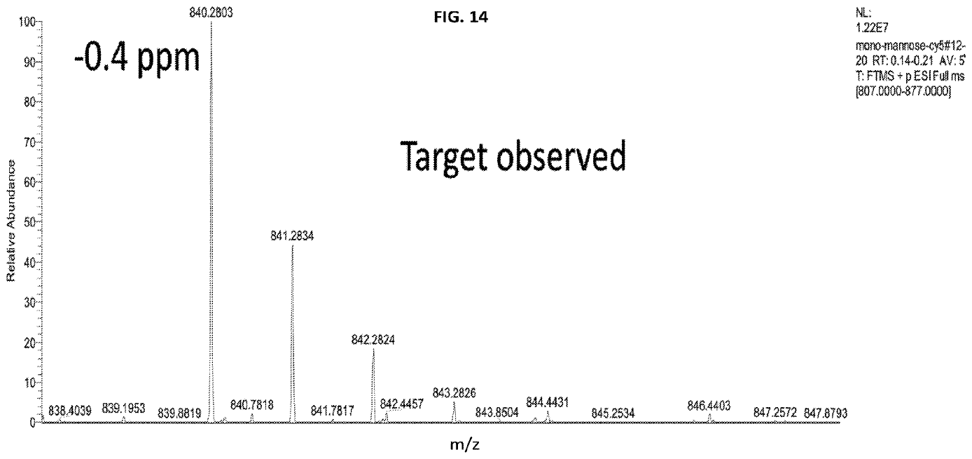

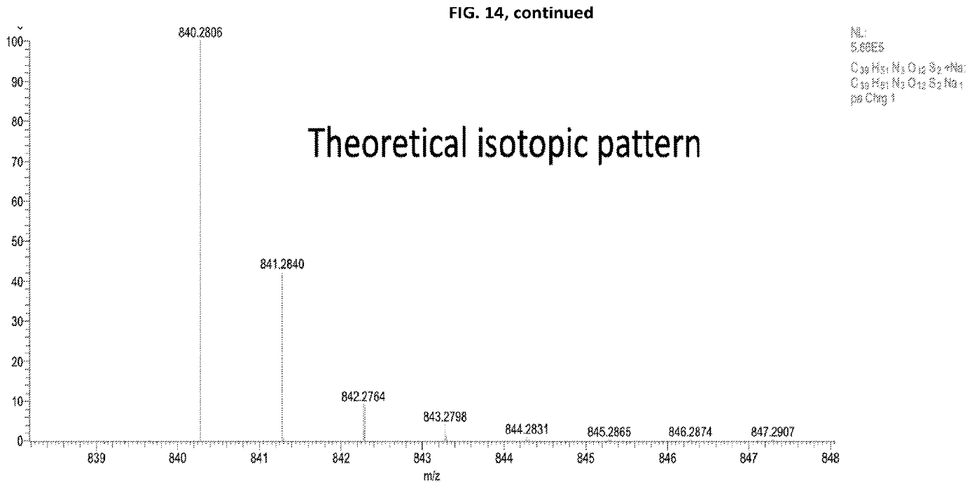

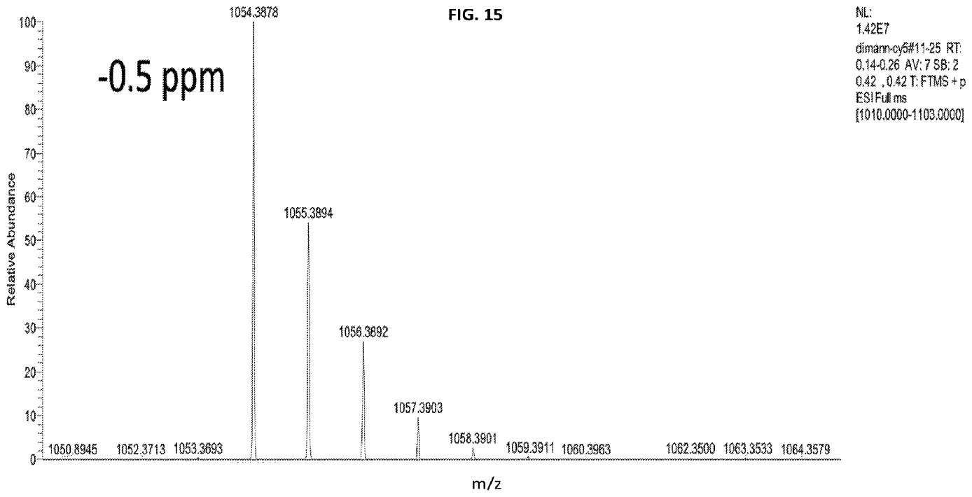

To a solution of D-mannosamine 1 (5 mg) in DMSO (1 mL) was added triethylamine (10 μL) and stirred for 20 min, followed by a solution of compound 2 Cy5-NHS (10 mg, 1 equiv.) in DMSO (1 mL). The reaction was stirred for another 1 hour at room temperature. The reaction was filtered to remove the solid and subjected to the HPLC separation to provide MR1-cy5 (3.7 mg, 35%). High-resolution MS: 840.2803 (M+Na, cal. 840.2806). High-resolution mass spectrum is shown in FIG. 14.

### Example 2. Preparation of Mann2-DTPA-Gd

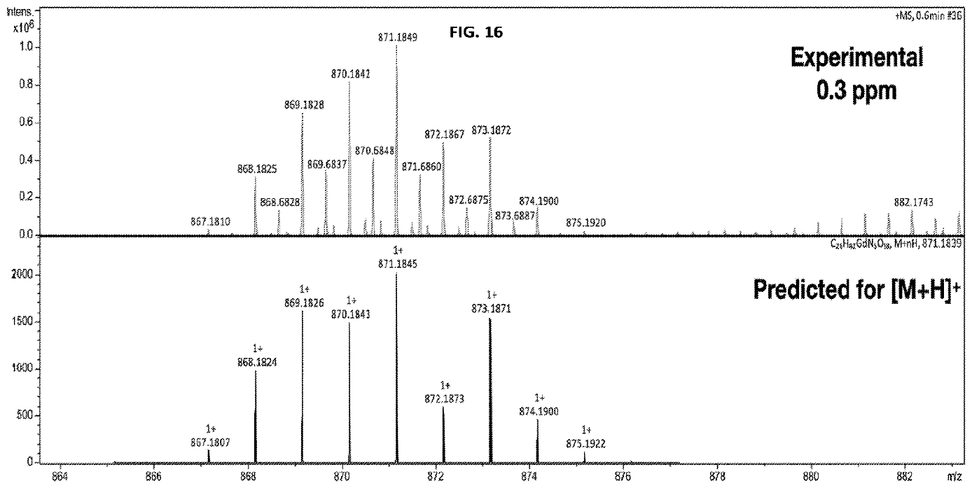

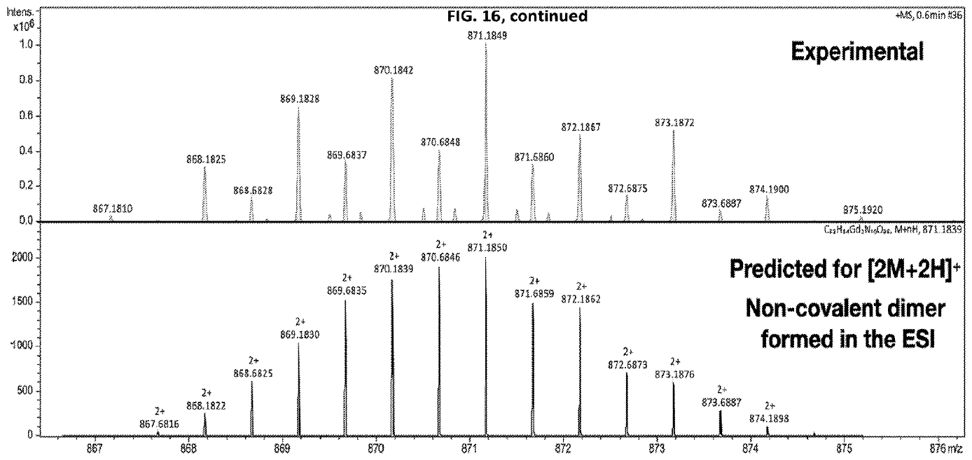

To a solution of D-mannosamine 1 (472 mg, 2.2 equiv.) in DMSO (6 mL) was added triethylamine (700 μL, 5.0 equiv.) and stirred for 30 min, then DTPA dianhydride 3 (318 mg, 1.0 equiv.) was added portionwise and stirred for another 3 h. The reaction underwent reversed column to give compound Mann2-DTPA 3 (460 mg, 64%). LCMS found m/z: 716.2 (M+H). A solution of the above compound 4 (286 mg, 1.0 equiv.) in water was mixed with a solution of GdCl3 (148 mg, 1.0 equiv.) in sodium ascorbate butter (pH 5.5) and stirred for 1 h. The reaction was filtered and HPLC separation gave the desired compound Mann2-DTPA-Gd (87 mg, 50%). High-resolution MS: 871.1849 (M+H, cal. 871.1845). The high resolution mass spectrum is shown in FIG. 16.

### Example 3. Preparation of MR2-cy5

### Step 1: Synthesis of Compound 7

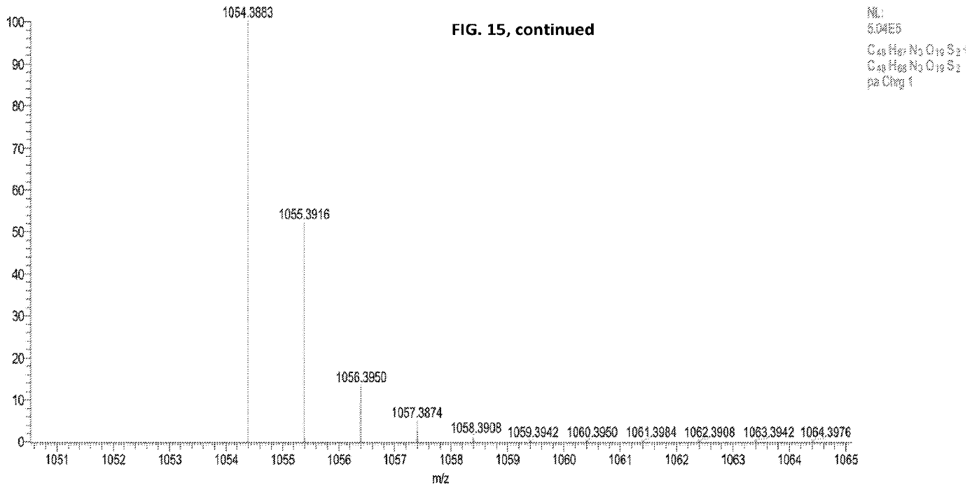

Compound 5 (360 mg, 1.0 equiv.), compound 6 obtained from bromination of 1,2,3,4,6-penta-O-benzoyl-alpha-D-mannopyranose (2.32 g, 2.2 equiv.) and activated 4 Å molecular sieves (1.0 g) in dichloromethane (12 mL) was stirred for 10 min. Then silver triflate (946 mg, 2.3 equiv.) was added to the reaction mixture at 0° C. The reaction was warmed to room temperature and stirred for another 2 h to complete. Saturated NaHCO₃ (5 mL) was added to the reaction and filtered. The filtrate was washed with brine, concentrated, dried, and underwent flash chromatography (gradient of hexane/ethyl acetate: 100% to 40%) to give the desired compound 7 (1.43 g, 65%) as a white powder. 1H-NMR (500 MHz, CDCl₃) δ: 8.10 (d, 4×1H, J=8 Hz), 7.99 (d, 4×1H, J=7.5 Hz), 7.93 (d, 4×1H, J=8 Hz), 7.80 (d, 2×1H, J=7.0 Hz), 7.76 (d, 2×1H, J=7.5 Hz), 7.59-7.52 (m, 4H), 7.45-7.30 (m, 17H), 7.29-7.20 (m, 9H), 6.15 (t, 2×1H, J=10 Hz), 5.90 (dd, 2×1H, J1=10 Hz, J2=3 Hz), 5.75 (d, 2×1H, J=12 Hz), 5.48 (d, 1H), 5.22-5.14 (m, 4H), 4.76 (d, 2×0.5H, J=11.5 Hz), 4.69 (m, 2×0.5H, J=10.5 Hz), 4.57 (d, 2×0.5H, J=12 Hz), 4.54-4.48 (m, 3H), 4.29 (b, 1H), 4.10-4.07 (m, 1H), 3.94 (d, 2×0.5H, J=11.5 Hz), 3.79-3.76 (m, 1H). 13C-NMR (125 MHz, CDCl₃) δ: 166.11, 166.08, 165.44, 165.39, 165.23, 155.96, 136.15, 133.36, 133.32, 133.25, 133.07, 133.01, 132.96, 129.83, 129.75, 129.71, 129.68, 129.20, 129.16, 128.89, 128.85, 128.82, 128.52, 128.48, 128.41, 128.40, 128.33, 128.29, 128.21, 98.26, 98.08, 70.16, 70.07, 69.93, 69.32, 69.25, 67.59, 67.14, 66.98, 66.59, 62.70, 49.99. LCMS found m/z: 1382.4 (M+H). The high resolution mass spectrum is shown in FIG. 15.

### Step 2. Synthesis of Compound 8

To a solution of compound 7 (1.1 g, 1.0 equiv.) in ethyl acetate and ethanol (v/v: 1/1, 5 mL) was added Pd/C (palladium on carbon, 110 mg) and stirred vigorously with a hydrogen balloon overnight. The reaction mixture was filtered and concentrated to provide compound 8 without further purification. 1H-NMR (500 MHz, CDCl₃) δ 8.10 (d, 2×1H, J=3.5 Hz), 8.09 (d, 2×1H, J=3.5 Hz), 7.95 (d, 2×1H, J=7.5 Hz), 7.91-7.89 (m, 6H), 7.73 (d, 2×1H, J=7 Hz), 7.69 (d, 2×1H, J=7.5 Hz), 7.53-7.46 (m, 4H), 7.40-7.35 (m, 6H), 7.34-7.29 (m, 4H), 7.27-7.22 (m, 4H), 7.19-7.16 (m, 4H), 7.14-7.10 (m, 2H), 6.21 (t, 2×1H), 5.96 (dd, 2×1H, J1=10 Hz, J2=3 Hz), 5.89 (m, 2H), 5.29 (d, 2×1H, J=19.5 Hz), 4.83-4.80 (m, 2H), 4.64 (d, 2×1H, J=10 Hz), 4.55-4.50 (m, 2H), 4.40 (d, 1H, J=7.5 Hz), 4.34 (dd, 1H, J1=10 Hz, J2=4 Hz), 4.17-5.10 (m, 3H), 3.33 (b, 2H). 13C-NMR (125 MHz, CDCl₃) δ 166.11, 165.99, 165.92, 165.50, 165.30, 165.23, 133.29, 133.16, 133.08, 132.95, 129.85, 129.77, 129.73, 129.66, 129.00, 128.88, 128.85, 128.73, 128.68, 128.41, 128.33, 128.24, 128.16, 98.37, 98.31, 70.76, 70.56, 69.95, 69.73, 69.46, 66.02, 65.78, 65.48, 62.45, 62.36, 50.97. LCMS found m/z: 1248.2 (M+H).

### Steps 3,4. Synthesis of Compound MR2-cy

To a solution of compound 8 (20 mg, 1.2 equiv.) in DMSO (0.6 mL) was added triethylamine (10 μL) followed by a solution cy5-SE (10 mg, 1.0 equiv.) in DMSO (0.6 mL). The reaction was stirred at room temperature for 2 h and monitored by LC-MS to give the major product 9. Water (10 mL) was added to the reaction and the precipitate was filtered, washed and put to the next step without further purification. To a suspension of the above precipitate in methanol (10 mL) was added a solution of sodium methoxide (25% weight in methanol, 1.1 equiv. to compound 8). The reaction was stirred at room temperature for 4 h. Minimum water was added to the reaction to dissolve any suspension and the solvent was removed under vacuum. The remainder was subjected to HPLC to afford the desired compound MR2-cy5 in the yield of 31% for two steps. High-resolution MS: 1054.3878 (M+H, cal. 1054.3883).

### Example 4. Preparation of MannGdFish

### Step 1. Preparation of Compound 11

To a solution of compound 8 (374 mg, 1.0 equiv.) in DMSO (3 mL) was added N, N-diisopropylethylamine (130 μL, 2.5 equiv.) and subsequent compound 10 (138 mg, 1.0 equiv.). The reaction was stirred at 70° C. overnight. The reaction underwent reversed phase column (gradient of water/acetonitrile: 95/5 to 0/100) to give compound 11 (363 mg) as a white solid in the yield of 71%, 1H-NMR (500 MHz, DMSO) &: 8.76 (b, 1H), 8.05 (d, 4×1H, J=6 Hz), 7.90 (d, 4×1H, J=6.5 Hz), 7.85 (d, 4×1H, J=7 Hz), 7.70 (b, 4×1H), 7.64-7.44 (m, 16H), 7.37-7.23 (m, 8H), 6.02 (t, 2×1H, J=10 Hz), 5.85-5.70 (m, 4H), 5.36-5.32 (m, 2H), 4.73-4.57 (m, 6H), 4.41 (b, 1H), 4.03-4.01 (m, 2H), 3.90-3.77 (m, 3H), 3.53-3.36 (m, 12H), 3.04-2.82 (m, 10H), 2.69-2.63 (m, 2H), 2.39-2.35 (m, 1H), 1.98 (b, 1H), 1.81 (m, 1H). 13C-NMR (125 MHz, DMSO) δ 172.80, 172.28, 170.52, 170.44, 165.28, 164.96, 164.70, 164.60, 133.94, 133.63, 133.55, 129.55, 129.29, 129.18, 128.93, 128.86, 128.63, 97.45, 97.36, 97.27, 97.20, 70.47, 69.91, 69.85, 68.44, 68.40, 67.04, 65.88, 62.62, 62.08, 55.37, 55.28, 54.37, 51.05, 50.94, 50.59, 50.40, 49.71, 49.63, 47.92, 47.80, 46.95, 32.58, 32.46. LCMS found m/z: 1708.3 (M+H).

### Steps 2,3. Preparation MannGdFish

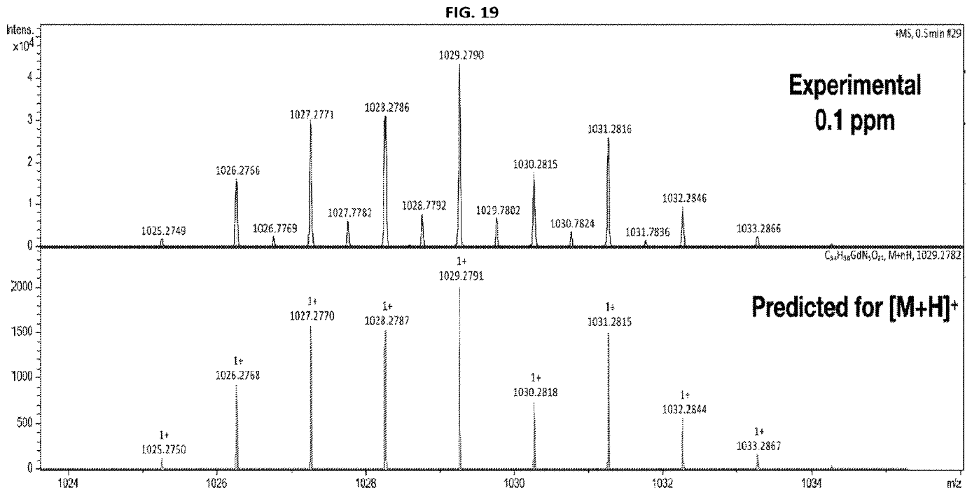

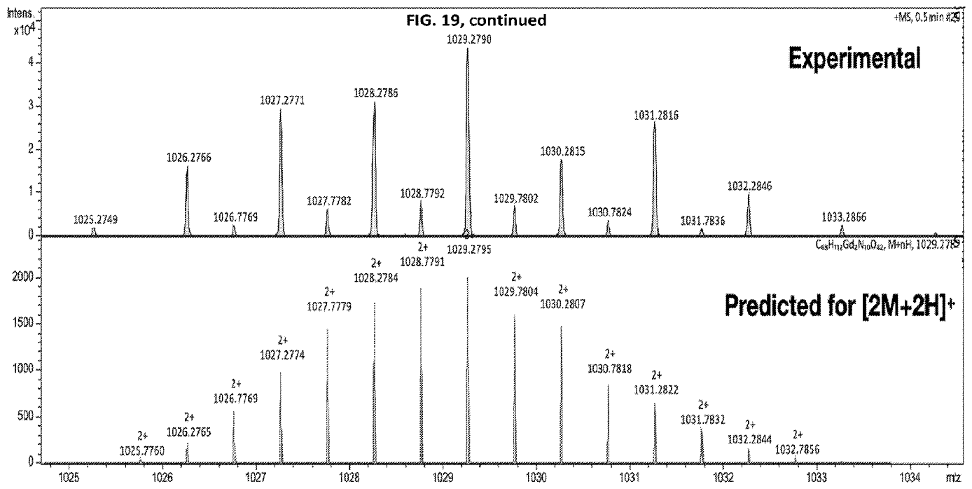

To a solution of compound 11 (256 mg, 1.0 equiv.) in methanol was added sodium methoxide (25% weight in methanol, 1.2 equiv.) and stirred for 3 h at room temperature. The reaction was adjusted to pH 7 with 1M HCl, concentrated to remove methanol, and the liquid phase was washed with ethyl acetate and lyophilized to give the deprotected compound without further purification. LCMS found m/z: 874.2 (M+H). A solution of the above compound was added to the solution of GdCl3 (56 mg, 1.05 equiv.) in sodium acetate buffer (pH 5.5, 4 mL). The reaction was stirred at room temperature for 1 h. The reaction underwent reversed phase column to give the desired compound MannGdFish (68 mg, 45% for two steps). High-resolution MS: 1029.2790 (M+H, cal. 1029.2791). The high resolution mass spectrum of MannGdFish is shown in FIG. 19.

An alternative procedure for the chelation step is to formulate the deprotected compound in 3M sodium acetate buffer (pH 4.5), then to add 68GaCl₃ eluate and heat the mixture for 90° C. for 10 minutes then cooled down to room temperature.

### BIOLOGICAL EXAMPLES

### Example 5. Fluorescence Imaging

M1- and M2-differentiated macrophages were incubated with 1/1000 dilution of MR2-cy5 stock solution (10 mM in DMSO) for 1 h at 37° C. and counter-stained with DAPI (4′,6-diamidino-2-phenylindole, Invitrogen). The cells were washed with PBS and fluorescence imaging was captured with a digital microscope (Nikon Eclipse TE2000-U).

### Example 6. ICP-MS

M1 and M2 differentiated macrophages were incubated with 1 mM of Mann2-DTPA-Gd for 1 h at 4° C. After washing with PBS (×3), cells were collected, digested with nitric acid (70%, 200 μL) overnight and subjected to ICP-MS to detect the amount of Gd.

### Example 7. Flow Cytometry

M1/M2 macrophages differentiated from RAW264.7 cells and M1-/M2-like macrophages obtained from wound tissues were stained and data were acquired on LSRII flow cytometer (BD Bioscience) and analyzed with BD FlowJo software (10.4).

### Example 8. MR Imaging

Eight-to nine-week-old C57BL/6J female mice were used for MRI of wound healing and glioma (Jackson Laboratory, ME). The generation of wound healing and glioma is described in Examples 14 and 15, respectively. The mice with subcutaneous wounds were imaged on days 1, 4, and 7 longitudinally or on day 7. Mice with glioma were imaged on the third week. All the mice were imaged pre- and at 0, 15, 30, 45 and 60 min after 0.3 mmol/kg of Mann2-DTPA-Gd or MannGdFish was administered intravenously via a tail vein using serial T1 rapid acquisition with relaxation enhancement (RARE) sequence (TR: 935.77 ms, TE: 13.59 ms, averages: 12, rare factor: 4, 256×256×48 matrix size, 0.156×0.156×1 mm³ voxel size) with chemical fat suppression using a Hermitian pulse shape with an 8.253 ms pulse and 701.19 Hz bandwidth 3.5 ppm down from the water peak and respiratory gating on a 4.7 T small animal MR scanner (Bruker, Cambridge, MA) with a 3 cm quadrature volume coil (Rapid MR International, Germany). Regions of interest (ROIs) were drawn and the contrast-to-noise ratios (CNRs, CNRpre-contrast subtracted) were calculated by a radiologist with over 20 years of experience blinded to the identity of the imaging agent used and the strain of the mice (n=3 per group).

### Example 9. Cytotoxicity of MannGdFish

The cytotoxicity of MannGdFish was evaluated using RAW264.7 cells (Passage 8 from the cell core of the Center for Systems Biology at Massachusetts General Hospital, Boston) with an MTT assay as described by Wang et. al (J Med Chem 64, 5874-5885 (2021)) (n=3).

### Example 10. Biodistribution, Retention, and Blood Half-Life of MannGdFish

6-10 weeks old of C57BL/6J mice were administered with MannGdFish intravenously 24 h after the wound injury. At 3 h and on day 7 after induction, mice were sacrificed and major organs including wound skins and the counter skins were harvested (n=3 for each time point). Blood samples were collected before and at different time points after the administration of MannGdFish (n=6) and centrifuged to obtain the plasma. The samples were weighed, treated with nitric acid, and subjected to inductively coupled plasma mass spectrometry (ICP-MS) to determine the amount of gadolinium as described in Example 6.

### Example 11. In Vitro Relaxivity of Mann2-DTPA-Gd/MannGdFish

Relaxation time (T1) of Mann2-DTPA-Gd/MannGdFish at concentrations of 0.1, 0.2, 0.3, 0.5, 0.6, 0.75, and 1 mM in PBS was measured on the Bruker Minispect (Bruker Analytics, MA) at 0.47 T (20 MHz) at 40° C. The slope value of the linear function of 1/T1 (s) to their corresponding concentrations is defined as the relaxivity (n=3 for each concentration).

### Example 12. In Vitro Specificity

M1 and M2 polarized differentiation. Raw 264.7 cells (Passage 8 from the cell core of center for Systems Biology at Massachusetts General Hospital, Boston) in 10 mL petri dishes were cultured with Dulbecco's modified eagle medium with high glucose (DMEM, Thermo Fisher Scientific, NY, USA) containing 10% of fetal bovine serum (FBS, Sigma-Aldrich, MO, USA) and 0.5% streptomycin/penicillin (Cellgro) until reaching 80% of confluency. Then differentiating media containing IFN-gamma (100 ng/ml the above medium) for M1 polarization and IL-4 (100 ng/mL) for M2 polarization were added to the dishes, respectively. After 24 h, the M1 and M2 polarized macrophages were ready for the following experiments.

### Example 13. Flow Cytometry

M1 and M2 differentiated macrophages were collected, centrifuged, and resuspended in PBS. For surface and intracellular staining anti-CD16/CD32, anti-CD11B APC/CY7, and anti-CD206 were obtained from Biolegend (San Diego, CA). Cells were spun, counted and resuspended in FACS buffer and incubated first with anti-CD16/CD32 to block Fc binding site for 20 minutes then washed with washing buffer for 3 times. Cells were then incubated with antibodies against surface markers for 30 min at 4° C. in the dark. For intracellular staining cells were then fixed and permeabilized using 1× Fix/Perm solution (BD Bioscience), washed in 1× permeabilization buffer (BD Bioscience), stained with anti-CD206-conjugated brilliant violet 711 for 30 minutes at 4° C. in the dark. Cells were subsequently washed and resuspended in FCS buffer. M2 differentiated cells were identified as CD11B+, CD206+ cells. Data were acquired on LSRII flow cytometer (BD Bioscience) and analyze with BD FlowJo software (10.4).

### Example 14. Subcutaneous Wound Healing

Wound healing procedure. Seven-to ten-week-old C57BL/6J female mice (The Jackson Laboratories, ME) and mannose-receptor deficient mice were used in this study. A circular outline on the back of a mouse was made with a 4 mm biopsy punch (Kai Medical, Japan) under anesthesia using isoflurane. Then a full-thickness excisional wound was generated that extended through the subcutaneous tissue. A silicone splint was glued over the wound, anchored with 6-0 nylon sutures, and covered with a transparent occlusive dressing (OpSite). Xylazine/norepinephrine (100 μL) were intraperitoneally injected twice a day for two days and the mice were monitored daily.

To isolate proinflammatory and anti-inflammatory macrophages from wound tissue, eight to twelve weeks old B6.129S4-Arg1tm1.1Lky/J mice (Jackson laboratory) were used. Wound model was generated as described above. Wound tissues were excised on days 1, 4, and 7. In brief, wound tissues were cut into small pieces and incubated for 1 hour at 37° C. in HBSS containing 0.7 mg/mL collagenase D (Milipore Sigma). Then the skin tissues were passed through 70 μm (BD Biosciences, San Jose, CA) strainer, and single cells were isolated from 44/67 Percoll (GE Healthcare, Boston, MA) gradient interface. For staining anti-CD16/CD32, anti-CD11B APC/CY7 and anti-CD86 APC were obtained from Biolegend (San Diego, CA). Cells were spun, counted, and resuspended in FACS buffer and incubated first with anti-CD16/CD32 to block Fc binding site for 20 minutes then washed with washing buffer for 3 times. Cells were then incubated with antibody against as mentioned above for 30 min at 4° C. in the dark. Cells were subsequently washed and resuspended in FCS buffer. Anti-inflammatory macrophages were identified as CD11B+, Arg1 YFP+, CD86-cells and proinflammatory macrophages were identified as CD11B+, CD86+, Arg1YFP-cells. Data were acquired and analyzed as described.

### Example 15. Mouse Model of Glioma

CT-2A-luc tumor cell culture followed a previously published protocol (N. Jalali Motlagh et al., Cancers (Basel) 13 (2021)). The cells were incubated at 37° C. with humidified air containing 5% CO2. Monolayer CT-2A-luc cells were cultured in DMEM supplemented with 10% FBS and 1% penicillin-streptomycin. To generate neurospheres, CT-2A monolayer cells were enzymatically dissociated by accutase (Stem Cell Technology, San Diego) and seeded in 25 cm² culture dishes at the cell concentration of 1×105 cells/mL in serum-free medium, composed of advanced DMEM/F12 medium (Life Technologies, Carlsbad, CA) with L-glutamine (2 mM; Cellgro, Manassas, VA), 1% N2 supplement (Life Technologies), 1% penicillin-streptomycin, recombinant EGF (20 ng/ml; R&D Systems, Minneapolis, MN), and recombinant FGF2 (20 ng/ml; Peprotech, East Windsor, NJ). After 10-11 days, the neurosphere CT-2A-luc (NS/CT-2A-luc) cells were collected, enzymatically dissociated with accutase, and prepared for intracranial injection.

Eight- to nine-week-old C57BL/6J female mice were used in this experiment (Jackson laboratory, ME). Dissociated NS/CT-2A-luc cells (7-8× 104) were implanted stereotactically into the brain (2.5 mm lateral and 1 mm anterior to Bregma and 3 mm deep) to generate an orthotopic intracranial tumor. The mice then were monitored daily for signs of discomfort or neurological symptoms.

### Other Embodiments

Although this disclosure contains many specific embodiment details, these should not be construed as limitations on the scope of the subject matter or on the scope of what may be claimed, but rather as descriptions of features that may be specific to particular embodiments. Certain features that are described in this disclosure in the context of separate embodiments can also be implemented, in combination, in a single embodiment. Conversely, various features that are described in the context of a single embodiment can also be implemented in multiple embodiments, separately, or in any suitable sub-combination. Moreover, although previously described features may be described as acting in certain combinations and even initially claimed as such, one or more features from a claimed combination can, in some cases, be excised from the combination, and the claimed combination may be directed to a sub-combination or variation of a sub-combination.

Particular embodiments of the subject matter have been described. Other embodiments, alterations, and permutations of the described embodiments are within the scope of the following claims as will be apparent to those skilled in the art. While operations are depicted in the drawings or claims in a particular order, this should not be understood as requiring that such operations be performed in the particular order shown or in sequential order, or that all illustrated operations be performed (some operations may be considered optional), to achieve desirable results.

Accordingly, the previously described example embodiments do not define or constrain this disclosure. Other changes, substitutions, and alterations are also possible without departing from the spirit and scope of this disclosure.

1. A compound of Formula (I): 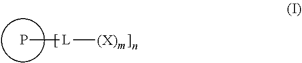
or a pharmaceutically acceptable salt thereof, wherein
P is a fluorescence imaging probe or a magnetic resonance imaging probe;
m is 1, 2, 3, 4, 5, 6, 7, 8, 9, 10, 11, or 12;
each X is independently X-1 or X-2: 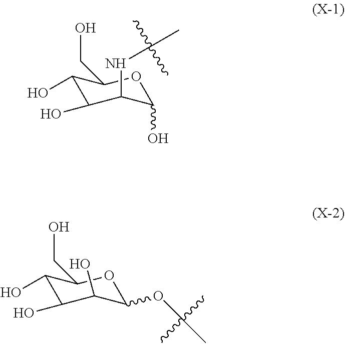 [SMILES: *NC1C(O)OC(CO)C(O)C1O ; *OC1OC(CO)C(O)C(O)C1O]
L is a C2-C20 alkylene; and
n is 1, 2, 3, 4, 5, or 6.

2. The compound of claim 1, wherein m is 1, 2, 3, 4, 5, or 6.

3. The compound of claim 1, wherein m is 1, 2, or 3.

4. The compound of claim 1, wherein L is a C2-C16 alkylene.

5. The compound of claim 1, wherein L is a C2-C12 alkylene.

6. The compound of claim 1, wherein L is 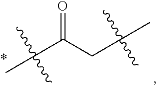 [SMILES: *CC(*)=O ; ","]
wherein * indicates the point of attachment to X.

7. The compound of claim 1, wherein L is 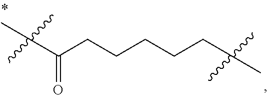 [SMILES: *CCCCCC(*)=O]
wherein * indicates the point of attachment to X.

8. The compound of claim 1, wherein L is 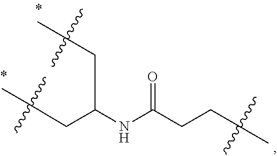 [SMILES: *CCC(=O)NC(C*)C*]
wherein * indicates the point of attachment to X.

9. The compound of claim 1, wherein L is 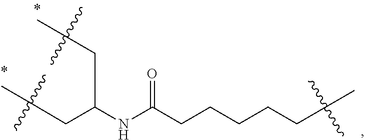 [SMILES: *CCCCCC(=O)NC(C*)C*]
wherein * indicates the point of attachment to X.

10. The compound of claim 1, wherein each X is X-1.

11. The compound of claim 1, wherein each X is X-2.

12. The compound of claim 1, wherein P is a fluorescence imaging probe.

13. The compound of claim 1, wherein P is

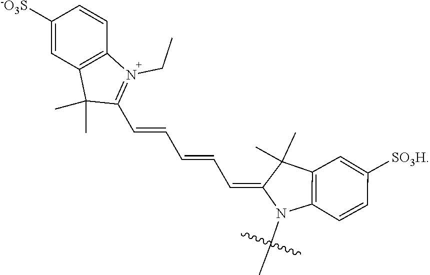

14. The compound of claim 1, wherein P is a magnetic resonance imaging probe.

15. The compound of claim 1, wherein P is

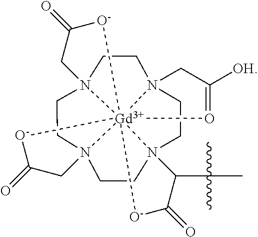

16. The compound of claim 1, wherein P is

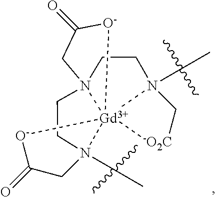

17. The compound of claim 1, wherein n is 1.

18. The compound of claim 1, wherein n is 2.

19. The compound of claim 1, selected from group consisting of: 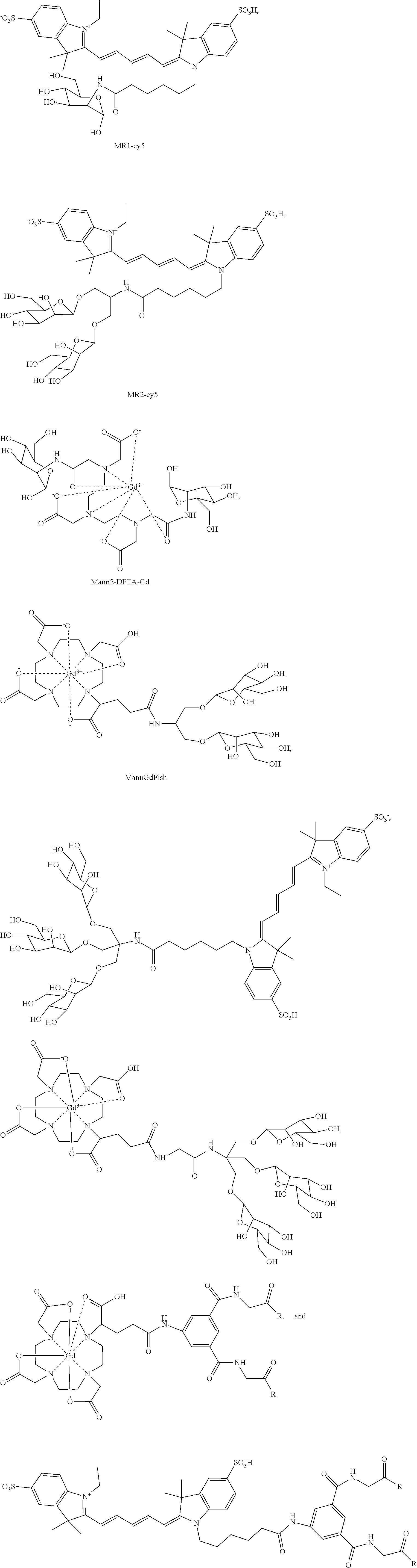
or a pharmaceutically acceptable salt of any of the foregoing, wherein

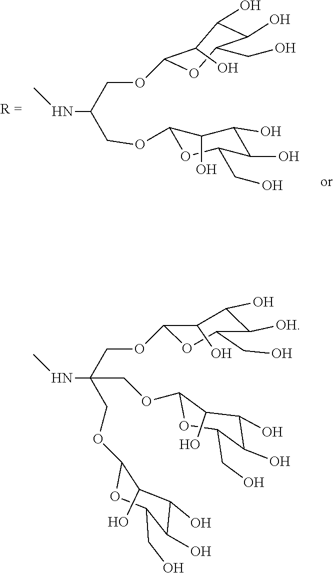 [SMILES: CNC(COC1OC(CO)C(O)C(O)C1O)COC1OC(CO)C(O)C(O)C1O ; CNC(COC1OC(CO)C(O)C(O)C1O)(COC1OC(CO)C(O)C(O)C1O)COC1OC(CO)C(O)C(O)C1O]

20. A pharmaceutical composition comprising a compound of claim 1, or a pharmaceutically acceptable salt thereof, and a pharmaceutically acceptable carrier.

21. A method of detecting M2-like macrophages in vitro, the method comprising:
(i) contacting a compound of claim 1 with cells or tissues;
(ii) waiting a time to allow the compound to accumulate in the cells or tissues; and
(iii) acquiring an image of the cells or tissues.

22. A method of monitoring M2-like macrophages in an organ or a tissue of a subject, the method comprising:
(i) administering to the subject an effective amount of a compound of claim 1, or a pharmaceutically acceptable salt thereof, or a pharmaceutical composition of claim 20;
(ii) waiting a time sufficient to allow the compound to accumulate in the organ or a tissue to be imaged; and
(iii) acquiring an image of the organ or the tissue of the subject,
wherein observing the image attributable to the compound of Formula (I) is indicative of M2-like macrophages in an organ or tissue.

23. A method of monitoring treatment of a disease or condition in a subject, the method comprising:
(i) administering to the subject an effective amount of a compound of claim 1, or a pharmaceutically acceptable salt thereof, or a pharmaceutical composition of claim 20;
(ii) waiting a time sufficient to allow the compound to accumulate in an organ or tissue of the subject affected by the disease or condition;
(iii) acquiring a first image of the organ or the tissue of the subject;
(iv) administering to the subject a therapeutic agent in an effective amount to treat the disease or condition;
(v) administering to the subject an effective amount of a compound of claim 1, or a pharmaceutically acceptable salt thereof, or a pharmaceutical composition of claim 20;
(vi) waiting a time sufficient to allow the compound to accumulate in the organ or tissue of the subject affected by the disease or condition;
(vii) acquiring a second image of the organ or the tissue of the subject; and
(viii) comparing the first and the second images,
wherein observing a difference between the first and the second images attributable to the compound of Formula (I) is indicative of progression of treatment of the disease or condition.

24. A method of diagnosing a disease or condition in a subject, the method comprising:
(i) administering to the subject an effective amount of a compound of claim 1, or a pharmaceutically acceptable salt thereof, or a pharmaceutical composition of claim 20;
(ii) waiting a time sufficient to allow the compound to accumulate in an organ or a tissue of the subject affected by the disease or condition; and
(iii) acquiring an image of the organ or the tissue of the subject,
wherein observing the image attributable to the compound of Formula (I) is indicative of the disease or condition.

25. A method of intraoperative imaging in a subject, the method comprising:
(i) administering to the subject an effective amount of a compound of claim 1, or a pharmaceutically acceptable salt thereof, or a pharmaceutical composition of claim 20;
(ii) waiting a time sufficient to allow the compound to accumulate in an organ or a tissue of the subject affected by the disease or condition; and
(iii) acquiring an image of the organ or the tissue of the subject,
wherein observing the image attributable to the compound of Formula (I) defines margins.

26. The method of claim 21, wherein the image comprises a fluorescence image.

27. The method of claim 21, wherein the image comprises a magnetic resonance image.
